(19) United States
(12) Patent Application Publication
(10) Pub. No.: US 2022/0313140 A1
(43) Pub. Date: Oct. 6, 2022

(54) ELECTROENCEPHALOGRAPHY NEUROFEEDBACK SYSTEM AND METHOD BASED ON HARMONIC BRAIN STATE REPRESENTATION

(71) Applicant: EEG Harmonics, LLC, Santa Barbara, CA (US)

(72) Inventor: Cana Selen Atasoy, Kidlington (GB)

(21) Appl. No.: 17/708,936

(22) Filed: Mar. 30, 2022

Related U.S. Application Data
(60) Provisional application No. 63/168,115, filed on Mar. 30, 2021.

(51) Int. Cl. A61B 5/377; G16H 40/63; A61B 5/16; A61B 5/055; A61B 5/00

(52) U.S. Cl. CPC A61B 5/377; G16H 40/63; A61B 5/165; A61B 5/055; A61B 5/0042

(57) ABSTRACT

A system and method for determining a type of sensory feedback to be applied to a subject. The system and method can include acquiring brain data, estimating from the brain data a plurality of harmonic brain states, determining a feedback variable based on the plurality of harmonic brain states, mapping the feedback variable to one or more types of sensory feedback, and applying the sensory feedback to the subject with one or more types of sensory feedback devices.

### RELATED APPLICATION

The present application claims priority to U.S. provisional patent application Ser. No. 63/168,115, filed Mar. 30, 2021, and entitled Electroencephalography Neurofeedback System And Method Based On Harmonic Brain State Decomposition, the contents of which are herein incorporated by reference.

### BACKGROUND OF THE INVENTION

Electroencephalography (EEG) is the measurement of brain activity that detects and measures brain waves by monitoring and recording the brain's electrical activity via electrodes placed on the scalp of a patient. More specifically, EEG measures voltage fluctuations resulting from electrical current within the neurons in the brain. EEG, by definition, is a measurement of the brain's spontaneous electrical activity over a period of time.

Typically, to measure brain waves of a person, the EEG electrodes are placed at various locations on the scalp. An electrode is an electrical conductor. Using the common-mode rejection method via differential amplification, the electrodes placed across a person's scalp detect electrical fluctuations. Traditionally, the International 10-20 system has been used to determine the proper number, placement and location of the electrodes on the scalp of the subject. The International 1-20 system is an internationally recognized method to describe and apply the location of scalp electrodes during the EEG acquisition. The predetermined electrode locations are evenly distributed over the scalp to optimize electrical activity detection taking place within the brain.

Conventional electroencephalographic neurofeedback (EEG-NFB) systems represent a broadly used brain training method aimed at developing selected self-regulation of brain activity using EEG. The EEG-NFB system involves a real-time EEG signal measurement, immediate data processing and feedback to the subject in real-time. Using such a feedback loop, the subject may gain better regulation or control of his/her neurophysiological parameters, by inducing changes in brain functioning of the subject and, consequently, mental states and behavior of the subject. The EEG-NFB can be used as a treatment for various neuropsychological disorders and can also improve cognitive capabilities, creativity, and relaxation in healthy subjects. The primary purpose of the EEG-NFB, particularly in the clinical environment, is to aid the subject in learning to better self-regulate their neurophysiological parameter(s) and consequently their mental state.

Specifically, the conventional EEG-NFB system is connected to the subject via the placement of electrodes on the subject's scalp. The electrodes sense and capture the EEG signals of the subject, and the data associated with the EEG signals is conveyed to an EEG-NFB system and associated hardware. The EEG-NFB system can analyze the data in real-time to determine appropriate sensory feedback information to be introduced back to the subject. Typical examples can include a video game, where the car's speed is controlled by some property of the subject's brain activity or a bar showing the intensity of the subject's brain activity, alongside a threshold, which the subject aims to achieve. When the threshold is reached, rewarding feedback (e.g., a pleasant auditory tone) can be given to the subject, reinforcing a desired mental state.

Current practices in EEG-NFB systems train a specific calculated variable (e.g., magnitude, polarization, phase relationship, or amplitude) of a specific electrode location or estimated voxels of the particular region of interest, based on the calculated feedback variable between two or more electrodes (e.g., active and reference electrodes). Yet it is well known that any neurological function or mental state is generated from the complex interaction of physiological neuronal processes at the whole brain level. Thus, the brain can best be understood as a complex system or network in which patterns of brain activity and the corresponding mental states emerge from the interaction between multiple physical (e.g., structural) and functional properties. Current state of the art in EEG-NFB systems, unfortunately, only use a rudimentary picture to generate the feedback information. None of the conventional EEG-NFB methods fully account for the overall picture of brain activity associated with a specific mental state. A reduced measurement of the brain's functioning fails to consider important variables of the overall processes involved in brain functioning, potentially compromising the quality and outcome of any corresponding neurofeedback treatment.

Another disadvantage of the current methods is that the temporal analysis of brain activity is done in a sub-optimal manner. The current EEG-NFB systems and associated methods either use a snapshot of the EEG signals at a given moment in time (i.e., no temporal analysis occurs), or the systems use a fixed set of time intervals to capture and store the EEG signals. The feedback variable is then calculated from this fixed time history. The length of the time interval is usually chosen arbitrarily. The resulting feedback loop thus suffers from unaccounted delays during processing and data transfer, which may render any feedback protocol void.

### SUMMARY OF THE INVENTION

The present invention is directed to a neurofeedback system for processing brain data, such as EEG data, and then determining therefrom a feedback variable. The feedback variable can be mapped to one or more sensory feedback devices forming part of a sensory feedback unit for exposing the subject to one or more types of stimuli.

Specifically, the neurofeedback system of the present invention is based on representing brain activity in terms of harmonic brain states (brain harmonics), which can be estimated in various different ways, as described herein, and therefore offers the advantage of considering multiple brain regions and their associated interrelations when estimating the neurofeedback. The neurofeedback system of the present invention can be used to aid the treatment of various neuropsychological disorders and can improve cognitive capabilities, creativity, and relaxation in the subject. Further, the neurofeedback system can be used in a clinical environment to support a subject in learning to self-regulate and to better manage certain neurophysiological parameters of their brain activity and thus more easily achieve certain desired mental states and consequently behaviors. The subject can learn how to enhance or inhibit specific electrophysiological parameter(s) through “operant conditioning” (i.e., the learning process in which a behavior's strength is modified using immediate feedback and positive reinforcement.

The present invention is directed to a method for determining a type of sensory feedback to be applied to a subject. The method can include acquiring brain data, estimating from the brain data a plurality of harmonic brain states, determining a feedback variable based on the plurality of harmonic brain states, mapping the feedback variable to one or more types of sensory feedback, and applying the sensory feedback to the subject with one or more types of sensory feedback devices. The method can further optionally include estimating a mental state of the subject based on the plurality of brain states and the feedback variable.

The method of the present invention further includes determining a brain connectivity matrix data indicative of a connectivity of the brain based on the brain data. In this regard, the method can determine the brain connectivity matrix data in one or more optional ways or approaches.

According to a first optional approach for determining the brain connectivity matrix data, the method includes applying one or more structural connectivity techniques to input MM data to estimate the physical white-matter inter-regional pathways in the brain based on the MRI type data, and generating output structural brain connectivity data. The method can also optionally apply a parcellation technique to the structural brain connectivity data to generate output parcellated brain connectivity data.

According to a second optional approach for determining the brain connectivity matrix data, the method includes applying one or more functional connectivity techniques to input functional MRI type data to estimate functional interrelationships between regions of the brain, and generating output functional brain connectivity data. The method can then optionally apply a parcellation technique to the output functional brain connectivity data to generate output parcellated brain connectivity data.

According to an optional third approach for determining the brain connectivity matrix data, the method includes applying one or more functional connectivity techniques to input EEG data, mapping the EEG data onto a cortical surface of the subject using a source location technique, estimating a functional connectivity of the brain by performing temporal correlations of the brain data over time, and generating output functional brain connectivity data. The method can then optionally apply a parcellation technique to the functional brain connectivity data to generate output parcellated brain connectivity data.

According to an optional fourth approach for determining the brain connectivity matrix data, the method includes applying one or more functional connectivity techniques to input EEG data, estimating a functional connectivity of the brain based on the EEG data free of source localization, and generating output signal connectivity data.

Further, the method of the present invention can estimate the plurality of harmonic brain states by determining, using a Laplace eigenvalue technique, the plurality of harmonic brain states based on the brain connectivity matrix data. Specifically, the harmonic brain states can be determined by determining an adjacency matrix data based on the brain connectivity matrix data, determining a graph Laplacian matrix data based on the adjacent matrix data, and decomposing the graph Laplacian matrix data to estimate the plurality of harmonic brain states. When decomposing the graph Laplacian matrix data to estimate the plurality of harmonic brain states, the method includes generating a plurality of eigenvectors indicative of the plurality of harmonic brain states, and generating a plurality of eigenvalues associated with the plurality of eigenvectors.

The method also includes determining a feedback variable based on the plurality of harmonic brain states by acquiring real-time EEG data, determining directly a plurality of EEG harmonic brain states associated with the EEG data, determining a distance between the plurality of EEG harmonic brain states and the plurality of reference harmonic brain states using a distance measuring technique to form a harmonic distance, and determining the feedback variable based on the harmonic distance.

The method of the present invention can also optionally determine the feedback variable based on the plurality of harmonic brain states by acquiring real-time EEG data; determining a plurality of harmonic brain states based on MRI, DTI, fMRI or EEG data prior to real-time EEG acquisition; decomposing the EEG data using a harmonic brain state decomposition technique to estimate a relative contribution of each of the plurality of harmonic brain states to the EEG data; determining a distance between the plurality of harmonic brain states' contributions to the real-time and reference EEG using a distance measuring technique to form a harmonic contribution distance; and determining the feedback variable based on the harmonic contribution distance.

The present invention is also directed to a system for determining a type of sensory feedback to be applied to a subject, comprising a brain connectivity estimation unit for estimating a brain connectivity matrix data indicative of a connectivity of the brain based on input brain data, a brain harmonic estimation unit for determining from the brain connectivity matrix data a plurality of harmonic brain states, and a feedback variable generation unit for determining a feedback variable based on the plurality of harmonic brain states. The system can also optionally include a mapping unit for mapping the feedback variable to one or more types of sensory feedback devices of a sensory feedback unit, wherein the sensory feedback device is configured to apply a sensory feedback to the subject, or a classification unit for estimating a mental state of the subject based on the plurality of brain states and the feedback variable.

The system of the present invention further includes determining a brain connectivity matrix data indicative of a connectivity of the brain based on the brain data. In this regard, the system can determine the brain connectivity matrix data in one or more optional ways or approaches.

According to an optional first approach for determining the brain connectivity matrix data, the system includes a structural connectivity unit for applying one or more structural connectivity techniques to the MRI data to estimate physical white-matter inter-regional pathways in the brain based on the MRI type data and for generating output structural brain connectivity data. The brain connectivity estimation unit further include an optional parcellation unit for applying a parcellation technique to the structural brain connectivity data to generate output parcellated brain connectivity data.

According to an optional second approach for determining the brain connectivity matrix data, the brain connectivity estimation unit comprises a functional connectivity unit for applying one or more functional connectivity techniques to the functional MRI type data to estimate functional interrelationships between regions of the brain and for generating output functional brain connectivity data. The brain connectivity estimation unit can include an optional parcellation unit for applying a parcellation technique to the output functional brain connectivity data to generate output parcellated brain connectivity data.

According to an optional third approach for determining the brain connectivity matrix data, the brain connectivity estimation unit comprises a functional connectivity unit configured for applying one or more functional connectivity techniques to the EEG data, mapping the EEG data onto a cortical surface of the subject using a source location technique, estimating a functional connectivity of the brain by performing temporal correlations of the brain data over time, and generating output functional brain connectivity data. The brain connectivity estimation unit can include an optional parcellation unit for applying a parcellation technique to the functional brain connectivity data to generate output parcellated brain connectivity data.

According to an optional fourth approach for determining the brain connectivity matrix data, the brain connectivity estimation unit comprises a functional connectivity unit that is configured for applying one or more functional connectivity techniques to the EEG data, estimating a functional connectivity of the brain based on the EEG data free of source localization, and generating output signal connectivity data.

The brain harmonic estimation unit can include an adjacency matrix unit for generating adjacency matrix data based on the brain connectivity matrix data. The brain harmonic estimation unit can also include a graph Laplacian determination unit for generating, based on the adjacency matrix data, graph Laplacian matrix data. The brain harmonic estimation unit can further include a decomposition unit for decomposing the graph Laplacian matrix data and for generating Laplace eigen vectors representative of the plurality of harmonic brain states and Laplace eigenvalues.

Once the harmonic brain states are determined, the feedback variable generation unit can be configured to directly determine a plurality of EEG harmonic brain states from real-time EEG data, determine a distance between the plurality of EEG harmonic brain states and the plurality of reference harmonic brain states using a distance measuring technique to form a harmonic distance, and determine the feedback variable based on the harmonic distance. According to another optional approach, the feedback variable generation unit can be configured to decompose real-time EEG data using a harmonic brain state decomposition technique to determine a plurality of harmonic brain states previously estimated from MRI, DTI, fMRI or EEG data in order to estimate a relative contribution of each of the plurality of harmonic brain states to the measured EEG data, determine a distance between the plurality of harmonic brain states' contributions to the real-time EEG data and the plurality of harmonic brain states' contributions to reference EEG data using a distance measuring technique to form a harmonic contribution distance, and determine the feedback variable based on the harmonic contribution distance.

### BRIEF DESCRIPTION OF THE DRAWINGS

These and other features and advantages of the present invention will be more fully understood by reference to the following detailed description in conjunction with the attached drawings in which like reference numerals refer to like elements throughout the different views. The drawings illustrate principals of the invention and, although not to scale, show relative dimensions.

FIG. 1 is a schematic block diagram of one example of a conventional neurofeedback system.

FIG. 2 is a schematic block diagram of a subsystem of the neurofeedback system of FIG. 1 employing a spatial distribution determination unit.

FIG. 3 is a schematic block diagram of a neurofeedback system according to the teachings of the present invention.

FIG. 4 is a schematic illustration of the decomposition of brain data into a series of harmonic brain states according to the teachings of the present invention.

FIG. 5 is a schematic data flow diagram of the brain connectivity estimation unit of the neurofeedback system of the present invention.

FIG. 6 is a schematic block diagram of the brain harmonic estimation unit of the neurofeedback system of the present invention.

FIG. 7 is a schematic flow chart diagram illustrating the methodology employed by the brain harmonic estimation unit of the present invention.

FIG. 8 is a schematic flow chart diagram illustrating a first embodiment of a methodology for determining a feedback variable employed by the neurofeedback system of the present invention.

FIG. 9 is a schematic flow chart diagram illustrating a second embodiment of a methodology for determining a feedback variable employed by the neurofeedback system of the present invention.

FIG. 10 is a schematic data flow diagram of the feedback variable generation unit of the neurofeedback system of FIG. 3 according to the teachings of the present invention.

FIGS. 11A-1, 11A-2, 11B-1, 11B-2, 11C and 11D are data flow and pictorial representations of the generation of brain harmonics from brain connectivity data as generated in FIG. 5 according to the teachings of the present invention.

FIG. 12 is a schematic diagram of an electronic device and/or associated system suitable for implementing the neurofeedback system of the present invention.

### DETAILED DESCRIPTION

Reference throughout to “one example” or “an example” or “for example” means that a particular feature, structure, or characteristic described in connection with the example is included in at least one example of the present disclosure or is discussed simply for illustrative purposes to promote understanding of the invention. Thus, the appearance of the phrases “one example” or “an example” in various places throughout this specification are not necessarily all referring to the same example. Furthermore, the particular features, structures, databases, or characteristics may be combined in any suitable combinations or sub-combinations in one or more examples. Also, it should be appreciated that the figures provided herewith are for explanation purposes to persons ordinarily skilled in the art and that the drawings are not necessarily drawn to scale.

The system and method of the present invention may be realized as system, apparatus, method, or computer program product. Accordingly, the present disclosure may take the form of an entirely hardware-comprised embodiment, an entirely software-comprised embodiment (including firmware, resident software, micro-code, etc.), or an embodiment combining both software and hardware aspects, that may all generally be referred to herein as a “circuit,” “module,” “unit” or “system.” Furthermore, embodiments of the present disclosure may take the form of a computer program product embodied in any tangible medium.

Any combination of one or more computer-usable or computer-readable media may be utilized. For example, a computer-readable medium may include one or more of a portable computer diskette, a hard disk, a random-access memory (RAM) device, a read-only memory (ROM) device, an erasable programmable read-only memory (EPROM or Flash memory) device, a portable compact disc read-only memory (CDROM), an optical storage device, and a magnetic storage device. Computer program code for carrying out operations of the present disclosure may be written in any combination of one or more programming languages. Such code may be compiled from source code to computer-readable assembly language or machine code suitable for the device or computer on which the code will be executed.

Embodiments may also be implemented in cloud computing environments. In this description and the following claims, “cloud computing” may be defined as a model for enabling ubiquitous, convenient, on-demand network access to a shared pool of configurable computing resources (e.g., networks, servers, storage, applications, and services) that can be rapidly provisioned via virtualization and released with minimal management effort or service provider interaction and then scaled accordingly. A cloud model can be composed of various characteristics (e.g., on-demand self-service, broad network access, resource pooling, rapid elasticity, and measured service), service models (e.g., software as a Service (“Saas”), Platform as a Service (“PaaS”), and Infrastructure as a Service (“IaaS”)), and deployment models (e.g., private cloud, community cloud, public cloud, and hybrid cloud).

The flowchart and block diagrams in the attached figures illustrate the architecture, functionality, and operation of one or more possible implementations of systems, methods, and computer program products according to various embodiments of the present disclosure. Each block in the flowchart or block diagrams may represent a module, segment, or portion of code, which comprises one or more executable instructions for implementing the specified logical function(s). It will also be noted that each block of the block diagrams or flow chart illustrations, and combinations of blocks in the block diagrams or flow chart illustrations, may be implemented by special purpose hardware-based systems that perform the specified functions or acts or combinations of special purpose hardware and computer instructions. These computer program instructions may also be stored in a computer-readable medium that can direct a computer or other programmable data processing apparatus to function in a particular manner, such that the instructions stored in the computer-readable medium produce an article of manufacture including instruction means which implement the function/act specified in the flowchart or block diagram block or blocks.

As used herein, the term “neurofeedback” or “EEG Neurofeedback” (EEG-NFB) is intended to mean a complete system, which measures the EEG signal of a subject (who receives the EEG-NFB) in real-time or near real-time and immediately processes the measured signals to extract one or more parameters of interest to provide a form of sensory feedback to the subject.

As used herein, the term “patient” or “subject” is intended to mean the person whose brain activity is being measured and to whom neurofeedback, such as for example EEG-NFB training/therapy or sensory input, is being administered.

As used herein, the term “mental state” is intended to mean a state of mind of the subject. The qualities of the mental state are relatively constant even though the state itself may be dynamic. Mental states comprise a diverse class including but not limited to anxious, depressed, relaxed, neutral, focused, distracted, sleep, meditative, under anesthesia, various other psychiatric conditions, and the like.

As used herein, the term “brain state” is intended to mean a pattern defined on the whole cortex, or on the scalp, or in the whole brain, or across the EEG electrodes, which can be used to represent the measured brain activity at a certain time.

As used herein, the term “feedback variable” is intended to mean a decimal, integer number, tensor, vector or matrix that constitutes a base variable for altering the properties of any sensory feedback applied to the subject. Examples of the feedback variable can include a single number that controls the volume of audio, a vector that changes the musical notes of the audio, a matrix that controls the intensity or colors of the pixels of an image or a video, and the like.

As used herein, the term “harmonic brain states” or “brain harmonics” is intended to mean a harmonic pattern of brain waves defined on the cortex, or on the scalp, or in the whole brain of the subject, or across the EEG electrodes, and which correspond to different harmonics or harmonic modes (i.e. eigenfunctions of the Laplace operator) that are applied to a selected structural or functional connectivity measure in the brain and can be estimated by the eigenvectors of the graph Laplacian applied to the chosen connectivity measure that is represented as a matrix, or can be estimated from any functional neuroimaging data or computed as the spherical harmonics and mapped to the cortex, scalp or the whole brain.

As used herein, the term “brain data” or “brain signals” or “brain waves” is intended to refer to any selected type of waves, information, data or signals that is associated with or measured, sensed or imaged from the brain of a subject. Examples of suitable types of brain data include electroencephalography (EEG) information, magnetic resonance imaging (MM) functional magnetic resonance imaging (fMRI) information, diffusion tensor imaging (DTI) data, and the like.

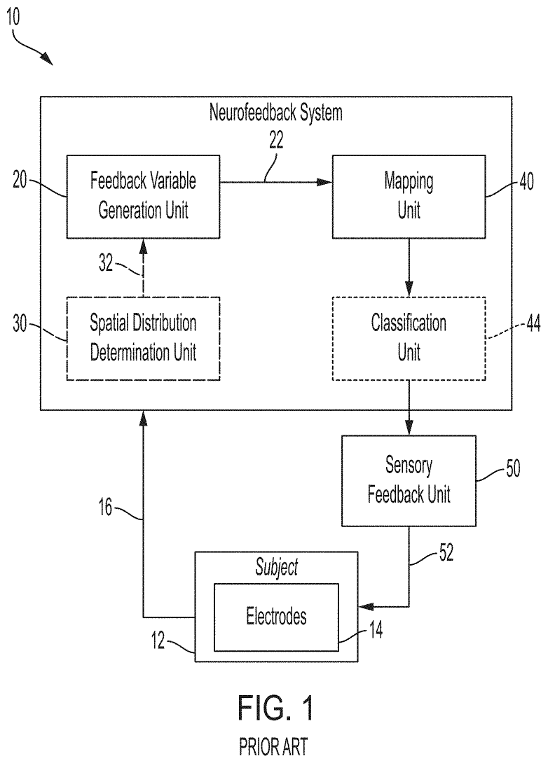

Neurofeedback systems for measuring brain data, such as electroencephalography (EEG) signals or data, of a subject 12 are known. One example of a conventional neurofeedback system 10 is illustrated in FIG. 1. A series of sensing electrodes 14 can be coupled to a scalp of the subject 12 to measure the brain signals of the subject. The electrodes 14 can be arranged in any selected configuration, and are preferably arranged in the International 10-20 electrode configuration. The electrodes 14 sense the ionic activity or current in the neurons of the brain and carry the sensed EEG signals 16 to the neurofeedback system 10. The EEG signals 16 can be directly received and processed by a feedback variable generation unit 20. The feedback variable generation unit 20 processes and analyzes the data associated with the EEG signals and then generates a feedback variable of interest. The feedback variable 22 can take any selected form and can be, for example, a decimal, an integer number, a vector, a matrix or a tensor. The feedback variable 22 thus includes selected information that is indicative of a type of sensory input to be applied to the subject in order to alter a mental state of the subject. The feedback variable 22 generated by the feedback generation unit 20 is conveyed to a mapping unit 40 for mapping the feedback variable 22 to one or more different types of sensory feedback application units or devices. For example, the mapping unit 40, based on selected parameters or values associated with the feedback variable 22, can activate one or more different types of sensory feedback units or devices and then activate the mapped device for applying selected types of sensory feedback information to the subject 12.

The mapping unit 40 can activate a mapped sensory feedback device which can include auditory devices, visual devices, tactile devices, or a combination thereof. In the current embodiment, the devices and units for applying or conveying the sensory feedback information to the subject is represented by the sensory feedback unit 50. The sensory feedback unit 50 can apply visual, audio, tactile, or other types of information, or combinations thereof, to the subject by corresponding units or devices, such as displays, speakers, tactile devices, and the like. The mapping unit 40 can activate a sensory information of type auditory, visual, tactile or a combination thereof. According to one embodiment, the mapping unit 40 can map the feedback variable 22 to a display unit for displaying images or video to the subject. Alternatively, the mapping unit 40 can generate an output mapping signal that is received by an audio device, such as a speaker, for applying selected types of audio signals to the subject. The audio output of the audio device can include audio of any selected type, duration, decibel level, and the like. The audio output can be random, intermittent, continuous, or variable. The mapping unit 40 can also generate an output signal that is received by a tactile device that can apply one or more different types of tactile signals to the subject 12. The output of the sensory feedback unit 50 forms the feedback signal 52 that is presented or applied to the subject 12. For example, different musical tones can be assigned to different values of the feedback variable 22 through the mapping unit 40 and the corresponding musical note can be played to the subject based on the current value of the feedback variable 22 at each time point, leading to a (near) continuous auditory information to be fed back to the subject based on certain aspects of their brain activity. The sensory feedback information in the form of the feedback signal 52 helps the subject modify, alter, or control one or more selected conditions related to the emphasis or reduction of certain types of mental states and their corresponding behaviors. Based on the teachings herein, those of ordinary skill in the art will readily recognize that the sensory feedback unit 50 can form part of the neurofeedback system 10 or can be a separate unit, as shown.

Further, the feedback variable 22 or the output of the mapping unit 40 can be processed by an optional classification unit 44 that can estimate or determine the subject's specific mental state based on the feedback signal or the mapping unit signal. Further, the classification unit 44 can optionally estimate or determine a desired mental state that the subject needs to attain in order to best address the actual mental state of the subject. The desired mental state can be calculated or determined by the classification unit 44 from the information associated with the EEG signals and the feedback variable, or the desired mental state can be preselected by a medical professional based on a subject's assessment.

The sensed EEG signals 16, which can be multi-channel EEG signals, can be directly processed and employed as biological baseline data by the feedback variable generation unit 20 of the neurofeedback system 10. The EEG signals can be acquired from the subject when the subject is disposed in a number of different conditions, such as for example in a resting brain state, or during task-performance or for event-related potentials (ERPs).

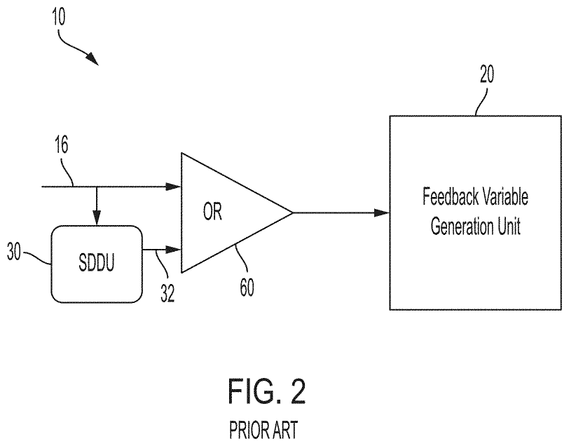

Alternatively, the EEG signals 16 can be processed by an optional spatial distribution determination unit (SDDU) and associated components prior to introduction to the feedback variable generation unit 20, as shown in FIGS. 1 and 2. The spatial distribution determination unit 30 can process the EEG signals 16 for estimating the actual spatial distribution of sources of the brain activity by applying an inverse solution thereto. The inverse solution applied by the spatial distribution determination unit 30 to the EEG signal 16 determines the source of the specific EEG signal in terms of the brain's neural activity. For example, the spatial distribution determination unit 30 can apply a nonparametric (e.g., distribution-free) inverse solution to the EEG signal 16. An example of a nonparametric inverse solution can include a minimum-norm formulation such as a Low-Resolution Brain Electromagnetic Tomography (LORETA) technique. Other inverse solutions can be estimated using beamforming or subspace. The spatial distribution determination unit 30 can generate an output spatial signal 32 having associated spacial data that can correspond to an estimated voxel of the source of the brain signal. A voxel represents a small patch of neurological tissue in three-dimensional space. The output spatial signal 32 of the spatial distribution determination unit 30 or the original input EEG signal 16 can be the input to the feedback variable generation unit 20. An optional selection unit 60 or methodology can be employed to pass the original EEG signal 16 or the spatial signal 32 to the feedback variable generation unit 20.

The illustrated feedback variable generation unit 20 can employ the sensed EEG signal 16 or the estimated voxel sources from the spatial distribution determination unit 30 to calculate or determine the feedback variable. In conventional systems, the feedback variable generation unit 20 is designed to employ multiple different techniques for calculating the feedback variable, including for example a slow cortical potentials (SCP) training technique, a coherence training technique, a frequency training technique, and the like. The conventional or prior art feedback variable generation unit 20 can employ the SCP training technique to detect shifts in the EEG signal 16 that reflect a depolarization of large groups of pyramidal cells in the cortex of the subject. Slow cortical potentials are negative or positive polarizations of the measured EEG signal lasting from about 300 ms to about several seconds. The slow shifts are caused by the burning of blood glucose that makes up the hemodynamics of the glial cells. These cells create large electro-negative swings when they depolarize. Sheets of cortical neurons are depolarized in the creation of the SCPs. In the prior art, the SCPs have been calculated from the measured EEG signal 16 and have been used in a threshold regulation mechanism to calculate the feedback variable 22. This method has been used, for example, to determine the feedback variable for subjects with attention deficit hyperactivity disorder (ADHD) to increase cortical negativity and subsequently improve attentional abilities. Also, the technique is used for subjects with epilepsy, targeting a decrease of cortical negativity power, hence increasing their threshold level for a seizure and reducing seizure likelihood.

The feedback variable generation unit 20 of the prior art has applied a coherence training technique to the EEG signal 16 or to the output spatial signal 32. The coherence training technique attempts to change the connectivity patterns among brain areas. In this context, coherence represents the degree of correlation between two or more different brain regions, based on the similarities in phase, amplitude, and frequency of the EEG signals.

The feedback variable generation unit 20 of the prior art has also applied a frequency training technique to the EEG signal 16 or to the output spatial signal 32. The frequency training technique aims to change the power of the brain activity within certain frequency bands. Traditionally, the EEG signals frequency bands are divided into delta (<4 Hz), theta (4-8 Hz), alpha (8-12 Hz), beta (13-30 Hz) and gamma (>40 Hz) frequency ranges. The rationale for this type of training is the proposed association between the amplitudes of specific frequencies and corresponding cognitive functions (i.e., frequency-to-function mapping).

In these prior art systems, during training, the subject's current state is estimated in real or close-to-real time. In normative-referenced neurofeedback (“Z-score neurofeedback), the current brain activity state is compared to the normal/desired state. In other feedback modalities, the brain activity state has either been “up-trained” (increased in amplitude, polarization, or measure of synchrony) or “down-trained” (inhibited in amplitude, polarization, or measure of synchrony). Sensory feedback was given to the user based on the differences between their brain activity versus a normative comparison or their brain activity versus their initial brain's dynamic baseline (taken throughout the training), respectively.

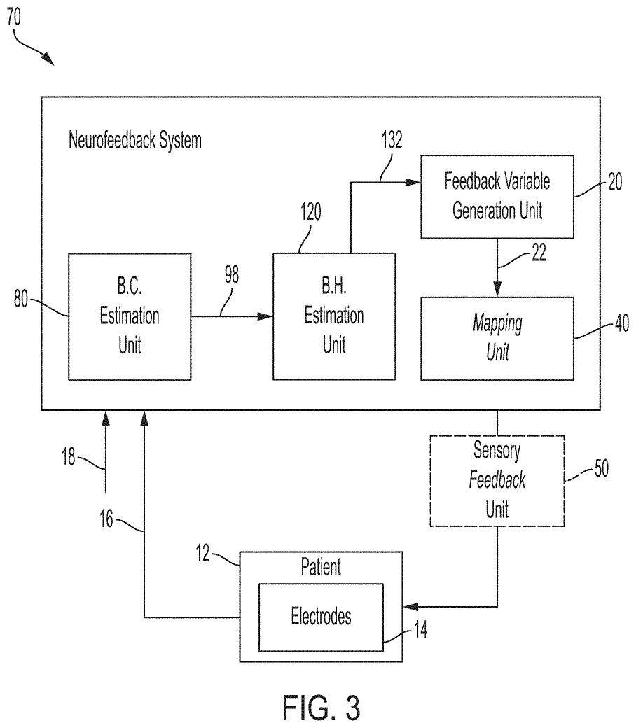

FIG. 3 illustrates an embodiment of the neurofeedback system 70 according to the teachings of the present invention. The illustrated neurofeedback system 70 employs harmonic brain states to estimate the feedback variable 22 by the feedback variable generation unit 20. Like numbers represent like parts throughout the various views. The neurofeedback system 70 can also be optionally used for the classification of mental states from the input brain data and from the calculated feedback variable 22. Specifically, the neurofeedback system 70 is based on representing brain activity in terms of harmonic brain states (e.g., brain harmonics), which can be estimated in various different ways, as discussed herein. As shown, a series of sensing electrodes 14 can be coupled to the scalp of the subject 12 in order to measure the brain signals thereof. The electrodes 14 can be arranged in any selected configuration, and are preferably arranged in the International 10-20 electrode configuration. The electrodes 14 sense the ionic activity or current in the neurons of the brain and carry the sensed EEG signals 16 to the neurofeedback system 70. The neurofeedback system 70 can process the EEG signals 16 and calculate therefrom a feedback variable 22 for use by a sensory feedback unit 50. The neurofeedback system 70 can also be configured to receive other types of brain data 18, in addition to the EEG data, including magnetic resonance imaging (MRI) data, diffusion tensor imaging (DTI) data, and functional magnetic resonance imaging (fMRI) data. The brain data can be used by the brain connectivity (B.C.) estimation unit 80 to generate the brain harmonics, in which the EEG data 16 is represented, as explained in detail herein. To this end, the EEG data 16 can be used as a signal input of the B.C. estimation unit 80 directly or can be filtered into one or multiple frequency bands, where each band's signal is represented in terms of brain harmonics.

The feedback variable 22 can be used to determine the sensory feedback to be introduced back to the subject or can also be optionally used to initially classify mental states of the subject from the measured brain activity, where the feedback can be estimated based on this classification. Specifically, the neurofeedback system 70, based on the EEG signals 16 and the feedback variable, can determine a mental state of the subject or directly estimate the suitable feedback to be presented to the subject (without classifying the subject's mental state) in order to aid the subject to achieve a desired mental state by representing brain activity, measured in EEG data, in terms of harmonic brain states (e.g., brain harmonics). In this process, the brain harmonics can be estimated in various different ways. Harmonic waves are ubiquitous in nature, emerging in various physical and biological phenomena. The present inventors have realized that harmonic brain states, estimated as the harmonic modes of the structural connectivity or the functional connectivity of the brain, may provide the elementary harmonic building blocks of brain activity and can be used in a neurofeedback system to provide selected sensory feedback to the subject. The structural connectome of a subject illustrating various examples of harmonic brain states or brain harmonics is shown for example in Atasoy, S. et al., Human Brain Networks Function In Connectome-Specific Harmonic Waves, Nature Communications, 2016, the contents of which are herein incorporated by reference. In the present invention, the harmonic brain states can be derived from structural data (e.g., MRI and DTI data), from functional data (e.g., fMRI data) or from EEG data, with our without applying the SDDU 30 to the EEG data and with or without applying a parcellation technique to any of these datatsets.

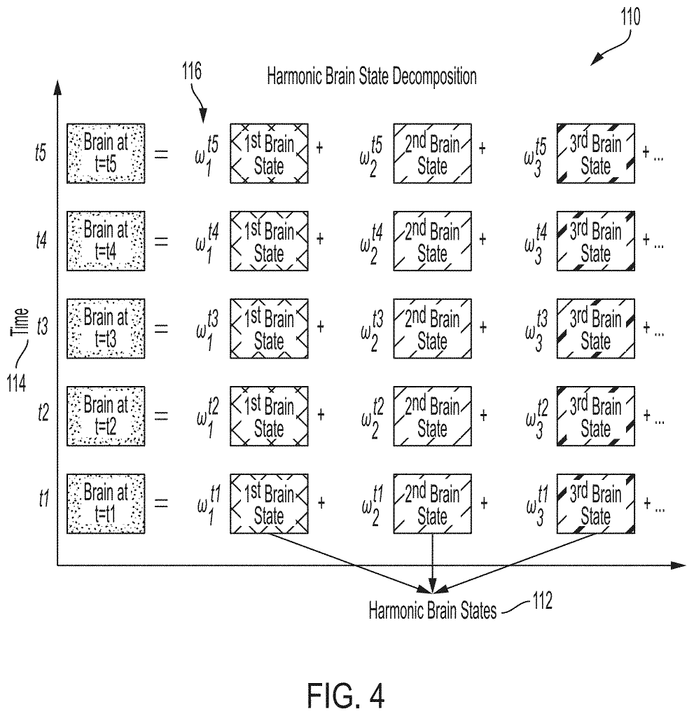

Furthermore, mathematically, the brain harmonics correspond to orthogonal functions and thus provide an orthogonal function basis, such that any pattern of brain activity can be described as a weighted sum of the harmonic brain states. Thus, using sets of harmonic brain states, the neurofeedback system 70 can reconstruct any pattern of brain activity by estimating the respective contribution of each harmonic brain state and summing up the harmonic patterns, weighted according to their contribution, as shown for example in FIG. 4. In FIG. 4, any pattern of brain activity, such as with the electrodes 14, can be decomposed into the harmonic brain states by estimating the activation/relative contribution of each harmonic brain state to represent the overall brain activity pattern. This decomposition is performed for the brain activity pattern of each time point in the EEG signal. As shown in the graph 110, and for illustration purposes only, three harmonic brain states 112 are shown and are decomposed over selected time periods 114, yet the decomposition can be done using various different numbers of harmonic brain states, where the maximum number is limited by the resolution of the structural or functional data from which they are estimated. For the harmonic brain state decomposition, the brain harmonics represent (decompose) the EEG data, and this can be achieved in various ways, examples of which are illustrated in FIGS. 11A-1, 11A-2, 11B-1, 11B-2, 11C and 11D. This process of estimating the relative contributions (strength of activation) of each of the harmonic brain states (e.g., harmonic brain state decomposition, as illustrated in FIG. 4) decodes the neural activity into a set of frequency-ordered harmonic brain states.

Further, each harmonic brain state can have an associated weight 116. In one embodiment, the present invention utilizes the weights 116 of the harmonic brain states within the measured EEG data 16 to implement an accurate neurofeedback system 70. This embodiment is referred to as a “Harmonic Decomposition Comparison” (HDC) technique. In yet another embodiment, the present invention optionally does not employ this harmonic brain state decomposition technique and instead utilizes directly the shape or other topological properties of the brain harmonics estimated from the EEG data 16 to estimate the feedback variable 22. This embodiment, without applying the harmonic brain state decomposition, is referred to as a “Direct Harmonic Comparison” (DHC) technique.

The present invention utilizes the harmonic brain states of the subject using the HDC and DHC techniques in order to implement an accurate neurofeedback system. According to the DHC technique, harmonic brain states (e.g., brain harmonics) are estimated directly from the EEG data acquired from the subject in a desired mental state (e.g., relaxed, meditative, neutral, sleep and the like) or from a desired population, such as neurotypical or experienced meditators and the like. This EEG data serves as a reference EEG data 26. During the neurofeedback process, the EEG data 16 of the subject who is to receive the sensory feedback is acquired by the electrodes 14 and the harmonic brain states are estimated from this new EEG data in real-time (real-time EEG data) for the EEG signals acquired in each time point or time window. The real-time neurofeedback is determined based on the distance between the harmonic brain states of the reference EEG data 26 and the real-time EEG data and is displayed to the subject as sensory feedback in real-time. This direct harmonic comparison technique introduces the concept of harmonic brain states estimated from the EEG data directly, which has not been done in conventional neurofeedback systems.

According to the HDC technique, both the reference EEG data 26 and the real-time EEG data 16 are decomposed using harmonic brain state decomposition techniques to estimate the relative contributions of each harmonic brain states, as illustrated in FIG. 4. In this approach, the harmonic brain states are previously computed (not in real-time) and can be estimated in various different ways, including for example dense structural harmonics, parcellated structural harmonics, dense functional harmonics, spherical harmonics mapped to the shape of the cortex or the scalp, and EEG harmonics. The real-time feedback variable to provide to the subject connected to the neurofeedback system 70 is then estimated using the distance between harmonic contributions of the real-time and reference EEG datasets.

Both techniques, such as the direct harmonic comparison technique and the harmonic decomposition comparison technique, incorporate harmonic brain states to represent EEG data in a neurofeedback system, and only differ in the way the feedback is estimated from harmonic brain states: in the former technique the feedback is estimated by comparing the EEG brain harmonics directly (e.g. their shapes, any topological measure applied to their shapes or their corresponding eigenvalues); whereas in the latter technique the feedback is computed using the comparison of the harmonic decompositions of the EEG reference and real-time EEG signals (e.g. using their contribution, the power of their contribution or the energy of their contribution or any measure applied to their relative contributions).

FIG. 3 illustrates the integration of brain harmonics (e.g., harmonic brain states) into the neurofeedback system 70. The illustrated neurofeedback system 70 of the present invention includes a brain connectivity (B.C.) estimation unit 80 for estimating the connectivity of the brain. As used herein, the term “brain connectivity” or “or “connectivity of the brain” is intended to mean or refer to a pattern of anatomical links (anatomical connectivity), or statistical dependencies (functional connectivity) or causal interactions (effective connectivity), between distinct units or parts of the brain. The units can correspond to small cortical patches, neuronal populations, anatomically segregated brain regions or areas, or EEG electrodes. The connectivity pattern is formed by structural links such as gray matter connections and/or white fiber pathways, or it represents statistical or causal relationships measured as cross-correlations, coherence, or information flow between the brain activities of the units. Hence, the brain connectivity encodes the structural or functional interrelations between different parts of the brain.

According to the present invention, different methods can be employed to implement each of the foregoing techniques for estimating brain connectivity. For structural brain connectivity, a dense structural brain connectivity approach or technique can be used by combining cortical surface connectivity estimated from brain data, such as MM data, with the long-distance white matter connectivity estimated from a different type of brain data, such as DTI data. For functional brain connectivity using fMRI data, again a dense functional brain connectivity approach or technique can be used by estimating correlations in a BOLD signal activity in the fMRI data, where the data is densely sampled from an fMRI volume or after mapping it to a cortical surface. For the dense EEG functional brain connectivity, the EEG signal measured from the scalp EEG electrodes can be mapped onto the cortical surface using one of many possible source location methods and the functional connectivity can be estimated in this space by simply taking temporal correlations across time courses. The dense connectivity approach can also be combined with a parcellation method by directly applying these parcellations to the dense connectivity matrices or the data from which they are estimated. The application of parcellation to the dense connectivity data results in parcellated brain connectivity data, which also can be used as the brain's connectivity. As an alternative to dense and parcellated versions of brain connectivity, the functional brain connectivity can also be estimated by utilizing the measured multi-channel surface EEG signal directly, without source localization, resulting in signal connectivity. Any chosen approach described herein, as well as other implementations or extensions of these structural and functional connectivity matrices, can be utilized to implement the brain's connectivity yielding connectivity matrix data, which can be used to estimate brain's harmonics.

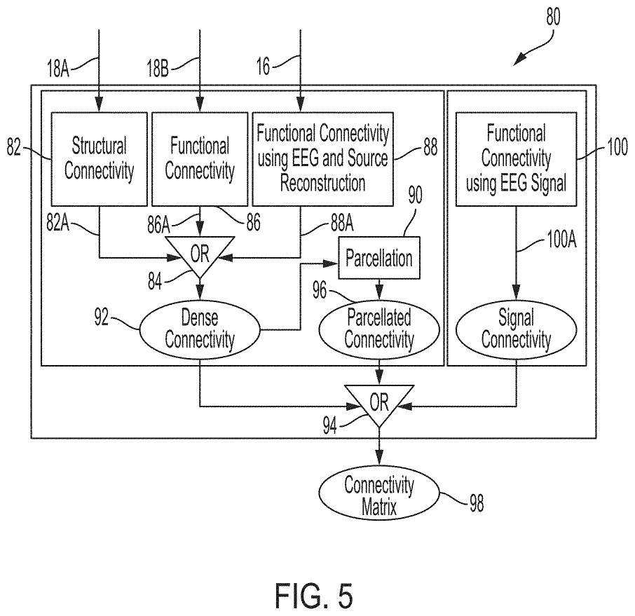

In the present invention, the brain connectivity estimation unit 80 can estimate the connectivity matrix data 98 from a variety of brain data sources, including but not limited to MRI data, DTI data, fMRI data and EEG data. As illustrated in FIG. 5, four possible optional or alternative techniques forming data pipelines for estimating or determining brain connectivity are shown and can include (i) structural brain connectivity from input MM or DTI data, (ii) functional brain connectivity computed from input fMRI data, (iii) functional brain connectivity computed from EEG data processed with the spatial distribution determination unit (e.g., source localization), and (iv) functional brain connectivity computed directly from the EEG signal 16. The techniques can be used solely or in any selected combination with each other.

The illustrated brain connectivity estimation unit 80 can include one or more units for determining the output brain connectivity matrix data 98. For example, the brain connectivity estimation unit 80 can include an optional structural connectivity unit 82 for applying one or more structural brain connectivity techniques to the brain data. In this regard, the structural connectivity unit 82 can receive structural brain data 18A, such as MRI data or DTI data, and process the data in a high resolution representation leading to dense structural connectivity data 82A encoding the interrelations between brain regions at a fine or high-resolution scale. The processing of MRI data and DTI data can be shown for example in FIGS. 11A-1 and 11A-2, which illustrates a processing pipeline for the brain's structural connectivity in terms of gray matter from MRI data 220A and white matter from DTI data 220B. The gray matter data 220A and the white matter data 220B are combined. The resultant dense connectivity matrix data 82A can be calculated from the combined data. The illustrated dense structural (connectome) harmonics 222 of the brain can be determined based on the dense connectivity matrix data 82A, as described herein. An example of this approach as illustrated in FIGS. 11A-1 and 11A-2 is described in the foregoing Atasoy, S. et al. publication.

Similarly, the brain connectivity estimation unit 80 can include an optional functional connectivity unit 86 to estimate the functional brain connectivity by processing input functional brain data 18B, such as fMRI data, that can be processed at a fine scale (e.g., high-resolution). The functional connectivity unit 86 can then generate dense functional connectivity matrix data 86A that encodes the functional interrelation between brain regions at a fine (high-resolution) scale. The processing of the fMRI data can be shown for example in FIGS. 11B-1 and 11B-2, which illustrates a processing pipeline for the brain's functional connectivity from resting state fMRI data 18B. The resultant dense functional connectivity matrix data 86A can be calculated from the fMRI data 18B. The illustrated dense functional harmonics 224 of the brain can be determined based on the dense functional connectivity matrix data 86A, as described herein. An example of this approach is described in Glomb, K. et. al., Functional Harmonics Reveal Multi-Dimensional Basis Functions Underlying Cortical Organization, Cell Reports, Aug. 24, 2021, the contents of which are herein incorporated by reference.

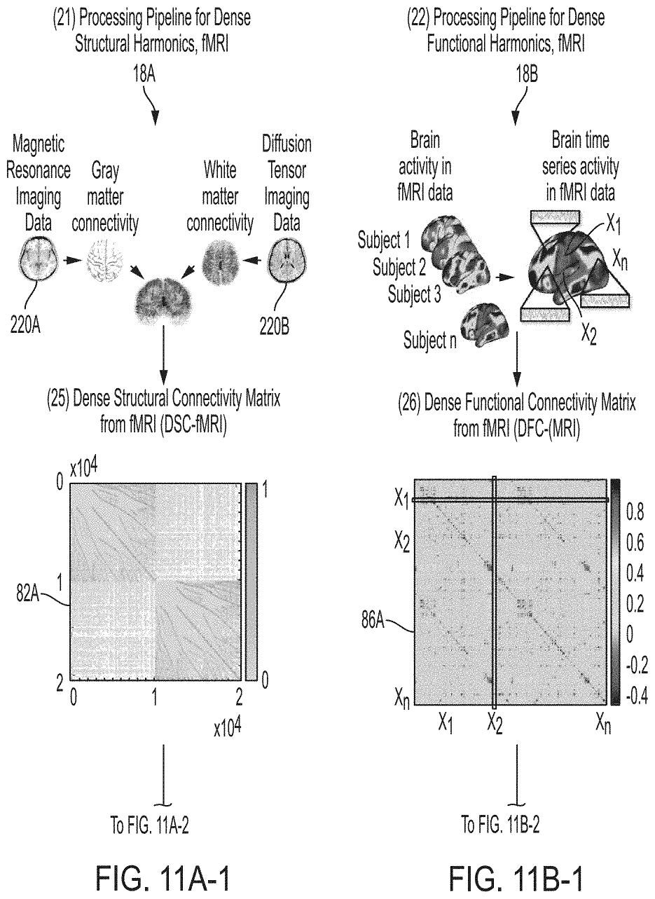

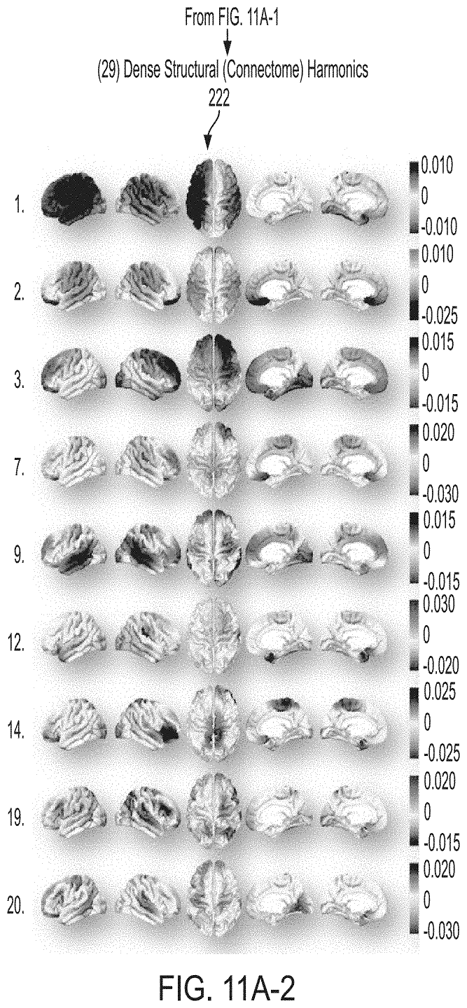

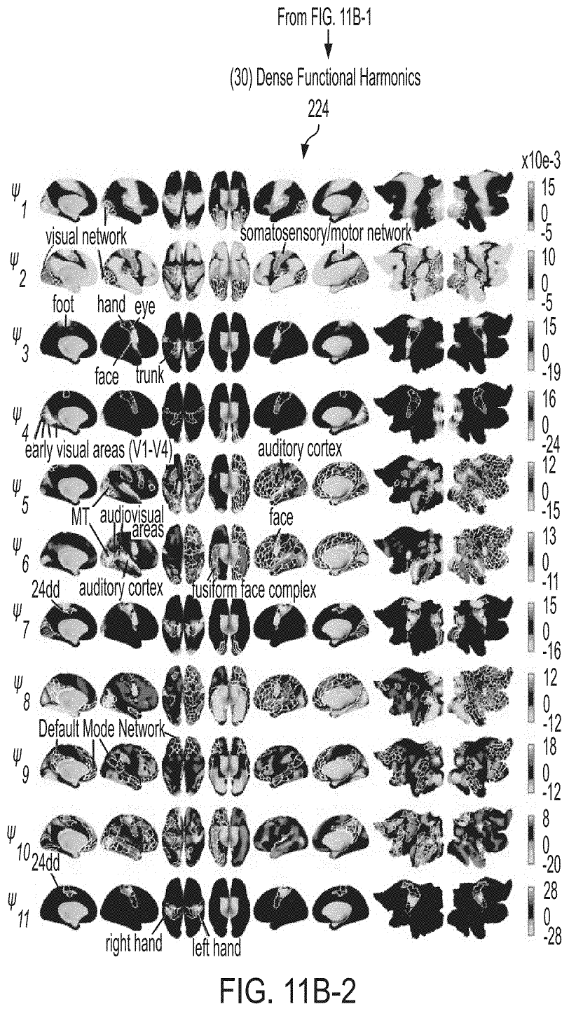

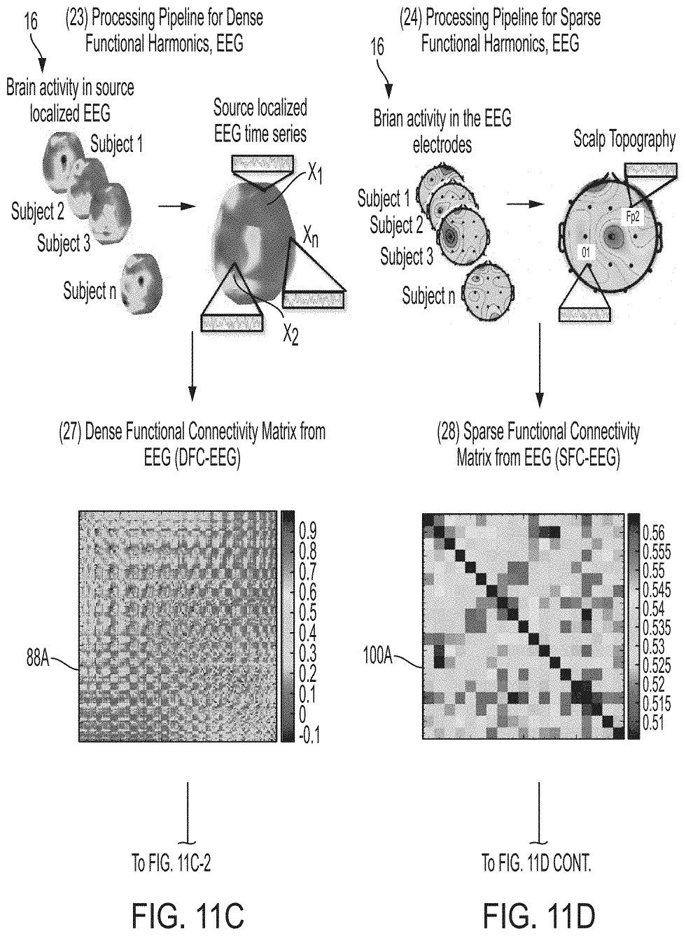

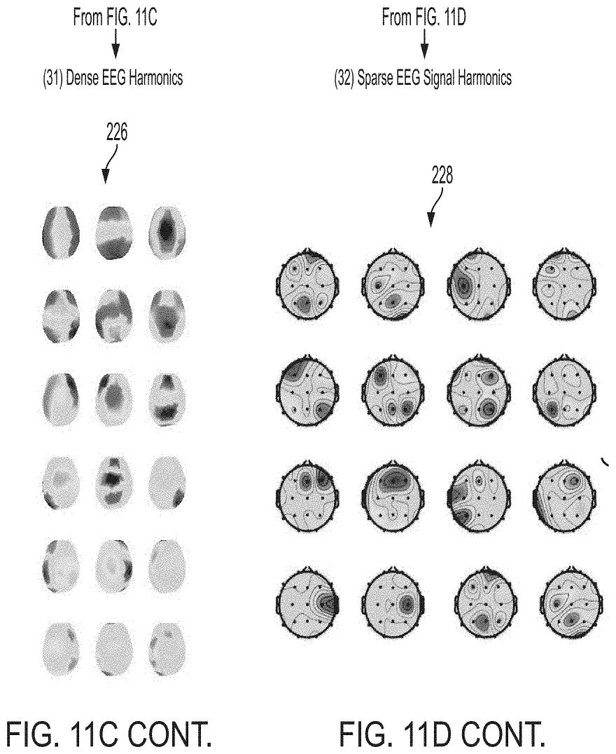

Alternatively, the illustrated brain connectivity estimation unit 80 can optionally include a second different functional brain connectivity unit 88 that receives and processes input EEG data 16, which can be further processed by the spatial distribution determination unit 30 for source localization. In this regard, the second functional brain connectivity unit 88 can apply a dense functional connectivity technique to the brain data by mapping the EEG signal onto a cortical surface of the subject using one of many possible source location methods utilized by the SDDU 30, and the functional connectivity can then be estimated in this space by simply taking temporal correlations across time courses or using any other connectivity measure. The resulting EEG functional connectivity matrix data 88A generated by the second functional connectivity unit 88 can be processed at a fine scale (e.g., high-resolution) leading to a dense EEG functional connectivity matrix data 92 that encodes the functional interrelation between brain regions extracted from the EEG data. The processing of the EEG data 16 can be shown for example in FIG. 11C, which illustrates an additional processing pipeline for the brain's functional connectivity from resting state EEG data. The resultant dense functional connectivity matrix data 86A can be calculated from the fMRI data 18B. The illustrated dense functional harmonics 226 of the brain can be determined based on the dense functional connectivity matrix data 86A, as described herein.

The brain connectivity estimation unit 80 can then optionally employ any type of selection methodology 84 to select one of the dense structural connectivity data 82A, the dense functional connectivity matrix data 86A, or the EEG functional connectivity matrix data 88A to form dense brain connectivity data 92 depending on the choice of the application or user. According to one embodiment, the chosen dense connectivity data 92 can be introduced to a parcellation unit 90. The parcellation unit 90 can apply a parcellation technique to the input or incoming brain connectivity data 92. The parcellation technique is a method or approach of segmenting sub-regions of a particular part of the brain. The parcellation unit 90 can then generate output parcellated brain connectivity data 96, which represents the connectivity information at a lower resolution, where the units correspond to different brain regions (parcels). The brain connectivity estimation unit 80 can choose to employ either the dense connectivity data 92 or the parcellated connectivity data 96 as the final connectivity matrix data 98. The illustrated brain connectivity estimation unit 80 can be utilized if the neurofeedback system 70 employs the harmonic decomposition comparison technique and associated pipelines. An example of brain harmonics using parcellated structural connectivity is described in Glomb, K. et. al., Connectome Spectral Analysis To Track EEG Task Dynamics On A Subsecond Scale, Vol. 24(3), pgs. 277-293, Neurolmage 221, Jul. 9, 2020, the contents of which are herein incorporated by reference.

As a further alternative, if the direct harmonic comparison technique is selected by the user, the brain connectivity estimation unit 80 can optionally employ a third functional connectivity unit 100 to directly estimate the brain connectivity from the input EEG signal, such as the EEG data 16. The third functional connectivity unit 100 does not apply EEG source localization, as done by the second functional connectivity unit 88, by applying the SDDU 30 to the EEG signal 16. This third functional connectivity unit 100 can then generate a connectivity matrix based on the EEG signal directly and encodes the signal connectivity to form the sparse signal connectivity matrix data 100A. Suitable EEG preprocessing techniques, such as artifact or motion removal can be applied to the EEG data 16. The EEG data 16 can be used as a single signal or can be filtered into one or more frequency bands of interest prior to estimating the signal connectivity. The processing of the EEG data 16 can be shown for example in FIG. 11D, which illustrates an additional processing pipeline for the brain's functional connectivity from input EEG data. The resultant sparse functional connectivity matrix data 100A can be calculated from the EEG data. The illustrated sparse functional harmonics 228 of the brain can be determined based on the sparse functional signal connectivity matrix data 100A, as described herein.

Depending on the chosen setting of the present invention, the brain connectivity estimation unit 80 can utilize one of the dense connectivity matrices 82A, 86A or 88A or one of their parcellated versions, such as the parcellated connectivity data 96, or the sparse signal connectivity matrix data 100A, as the final connectivity matrix data 98. An optional selection method or technique 94 can be employed to select and to pass the output data that functions as the final connectivity matrix data 98 for the brain connectivity estimation unit 80. The connectivity matrix data 98 can be subsequently employed to estimate the brain harmonics (e.g., harmonic brain states). The brain connectivity estimation unit 80 thus forms processing pipelines for generating the connectivity matrix data 98.

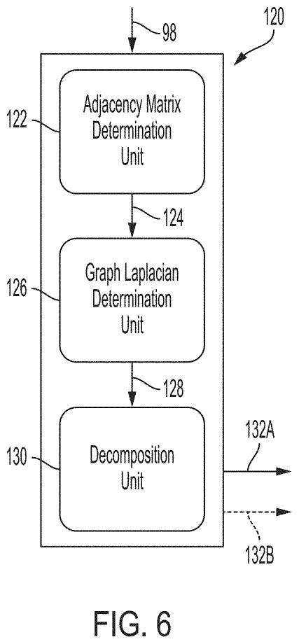

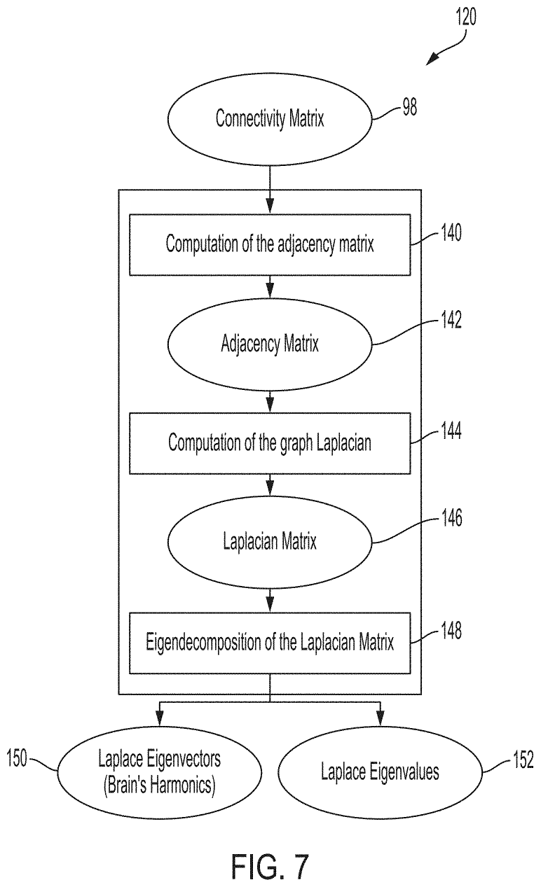

With reference to FIGS. 3 and 6-7, the illustrated neurofeedback system 70 also includes a brain harmonics (B.H.) estimation unit 120 for receiving the connectivity matrix data 98 and for generating, based thereon, harmonic brain states 132 that can be employed by the feedback variable generation unit 20 to determine the feedback variable 22.

The harmonic modes, which occur in various physical and biological phenomena, can be estimated by solving a Laplace eigenvalue problem forming part of a Laplace eigenvalue technique, as follows:

Δφᵢ=λᵢφᵢ, with 0≤i≤n, Eq. (1)

where the eigenfunctions φᵢ reveal the shape of the harmonic modes (stable harmonic waves/patterns) emerging on the particular domain on which the equation has been solved and the eigenvalues λᵢ relate to the frequency of the harmonic brain modes.

The brain harmonics estimation unit 120 can include an adjacency matrix determination unit 122 for processing the connectivity matrix data 98 and determining an adjacency matrix 124. Once the adjacency matrix is determined, the extension of Eq. (1) can be employed to estimate brain harmonics, which relies on the equivalent of the Laplace operator Δ defined for graph structures. This graph Laplacian can be applied to any connectivity matrix data 98 allowing for Eq. (1) to be solved on the domain corresponding to the particular types of brain connectivity forming part of the connectivity matrix data 98. This brain connectivity data can be structural or functional or related to any other measure leading to the brain connectivity matrix. As the first step for the application of the graph Laplacian to the particular type of brain connectivity matrix data 98, an adjacency matrix 124 can be calculated with the adjacency matrix determination unit 122 based on the connectivity matrix data 98. This step can be implemented by various methods. In one example, the method can include connecting each data point (index in the connectivity matrix) with its k-nearest neighbors according to the values in the connectivity matrix data and setting all other connections to 0. This can lead to the adjacency matrix 124 estimated by a k-nearest neighbors method or technique. The brain harmonics estimation unit 120 can thus calculate from the connectivity matrix data 98 the adjacency matrix 124, step 140. Another example can include applying a threshold to the connectivity matrix data 98 and setting all connections to settings weaker than the selected threshold to 0, which can lead to the adjacency matrix 24 estimated by thresholding, referred to as an epsilon-balls technique. The adjacency matrix 24 can be binary in nature (e.g., consisting of the values 0 and 1 only, where 0 indicates no connection and 1 indicates presence of a connection) or can be weighted, where the values in the adjacency matrix 124 indicate the strength of the connection between two points. Any method that takes the connectivity matrix data 98 as an input and yields an adjacency matrix 124 as an output can be utilized. Once the adjacency matrix 124 is estimated or determined via a selected method, step 142, the graph Laplacian can then be defined, step 144, as:

[symbol]

=D−A, Eq. (2)

where [symbol] denotes the graph Laplacian, A is the computed adjacency matrix 124 and D is the degree matrix computed from the adjacency matrix. The Degree Matrix D is a diagonal matrix defined as:

$$D := \{ \begin{matrix}{deg}_{i} & if i = j \\ 0 & otherwise ,\end{matrix}$$

with degᵢ being the number of all connections of the entry i. Various different implementations can be used for the calculation of the graph Laplacian leading to the graph Laplacian matrix, step 146, including but not limited to those incorporating different normalizations of the graph Laplacian [symbol] defined in Eq. (2). As such, the brain harmonics estimation unit 120 can employ a graph Laplacian determination unit 126 for processing the adjacency matrix data 124 and for generating therefrom the graph Laplacian matrix data 128.

The brain harmonics estimation unit 120 is also configured to include a decomposition unit 130 for receiving and processing the graph Laplacian matrix data 128 and decomposing this matrix 128 into its eigenvectors 132A and the corresponding eigenvalues 132B by solving its eigendecomposition as follows:

[symbol]

vᵢ=eᵢvᵢ, with 0≤i≤n, Eq. (3)

where vᵢ denotes the eigenvectors 132A of the graph Laplacian matrix 128 and eᵢ denotes the corresponding eigenvalues 132B, step 148. The resulting Laplace eigenvectors {eᵢ}ᵢ₌₀ⁿ, define the brain harmonics or harmonic brain states. Consequently, the decomposition unit 130 can decompose the input graph Laplacian matrix data 128 and generate therefrom a series of Laplace eigenvectors 132A and Laplace eigenvalues 132B that are indicative of the brain harmonics or harmonic brain states, steps 150 and 152. See Atasoy, S. et. al., Harmonic Brain Modes: A Unifying Framework For Linking Space And Time In Brain Dynamics, The Neuroscientist, Vol. 24(3), 2018, the contents of which are herein incorporated by reference. A more detailed description of the a decomposition technique is described in Atasoy, S., et al., Connectome-Harmonic Decomposition Of Human Brain Activity Reveals Dynamical Repertoire Re-Organization Under LSD, Scientific reports, 7.1 (2017), pgs. 1-18, the contents of which are herein incorporated by reference.

In summary, the type of harmonic brain states varies or changes based on the type or source of the connectivity matrix data 98. For example, the structural connectivity of the brain of the subject generated by the structural connectivity unit 82 can be used to estimate the brain's connectivity, where the cortical surface connectivity estimated from the input brain data (e.g., Mill data) with the long-distance white matter connectivity estimated from diffusion tensor imaging (DTI) data are combined, with or without applying a parcellation technique. The harmonic brain states can then be estimated from the dense structural connectivity matrix data as a series of dense structural (e.g., connectome) harmonics or from the parcellated structural connectome as a series of parcellated connectome harmonics.

Likewise, the functional connectivity of the brain generated by the functional connectivity unit 86 can be used to estimate the brain's connectivity, where the functional connectivity forms part of the connectivity matrix data 98 estimated from the input brain data (e.g., from resting state fMRI data). The harmonic brain states can then be estimated from the dense functional connectivity matrix data as a series of dense functional harmonics or similar to the dense structural connectivity, this dense functional connectivity can be combined with a parcellation method leading to a parcellated functional harmonics.

Similarly, the resting state EEG data after applying source localization (SDDU) and generated by the second functional connectivity unit 88 can be used to estimate the brain's functional connectivity using source localized EEG data. The source localized EEG data can form part of the connectivity matrix data 98. The harmonic brain states can then be determined from the connectivity matrix data 98 leading to dense EEG harmonics. Further, the brain's connectivity can be estimated by the third functional connectivity unit 100 by directly using the EEG signal as the input source data. The harmonic brain states can then be determined from EEG harmonics (e.g., connectivity matrix data) leading to sparse EEG harmonics. The parcellation technique applied by the parcellation unit 90 can be applied to any of the foregoing types of matrix data, as shown for example in FIG. 5.

The harmonic brain state data generated by the brain harmonic estimation unit 120 can include Laplace eigenvectors 132A and Laplace eigenvalues 132B, which can then be conveyed to the feedback variable generation unit 20 for generating the feedback variable 22. The feedback variable 22 can be conveyed to the sensory unit 50 for generating one or more selected types of stimuli for application to the subject 12.

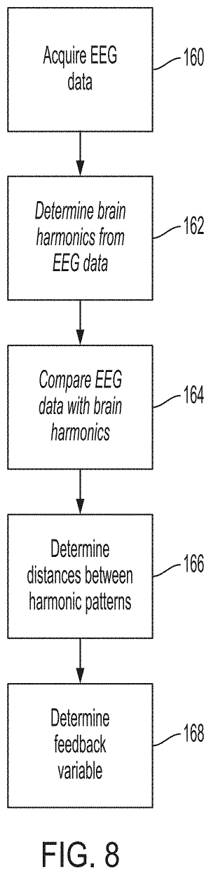

The feedback variable generation unit 20 can generate the feedback variable 22 based on the subject's brain activity as measured by the EEG electrodes 14 using two alternative approaches, methodologies or techniques, both of which employ a distance measuring technique. In the first approach, referred to herein as Feedback Estimated based on Direct Harmonic Comparison, the brain harmonics (harmonic brain states) are estimated directly from the EEG data 16 acquired from the brain harmonic data (reference EEG data) of the subject 12 or of a desired population such as neurotypical in a desired mental state (e.g. including but not limited to relaxed, meditative, neutral, and sleep) before the real-time neurofeedback application. As shown for example in FIGS. 3, 8 and 10, the brain data, such as real time EEG data 16 of the subject 12, can be captured, step 160. The brain harmonics (e.g., harmonic brain states) are then computed directly from the input EEG data 16 in real-time, steps 162 and 190. This yields real-time brain harmonics data, step 192. The feedback variable generation unit 20 then compares the brain harmonics of the real-time EEG data with the Laplace eigenvector information 132A (brain harmonics) generated by the brain harmonic estimation unit 120, step 164. The feedback variable generation unit 20 then computes or determines the distance between the brain harmonic patterns of the reference EEG data and the real-time EEG data using a distance measuring technique, steps 166 and 194. This yields a harmonic shape distance, step 196. The harmonic shape distance is then employed by the feedback variable generation unit 20 to generate the feedback variable 22, step 168. The distance measuring technique can be chosen to compare the particular shape or topology of the two different sets of harmonics (i.e., of the reference EEG data and the real-time EEG data), as well as incorporating their corresponding eigenvalues. This technique introduces the concept of determining or estimating harmonic brain states directly from the EEG data. As such, this technique involves the estimation of the brain's harmonics directly from the EEG data without involving any additional kind of functional or structural imaging, such as fMRI, DTI, and MRI data. The feedback variable 22 can then be determined from the measured harmonic distances.

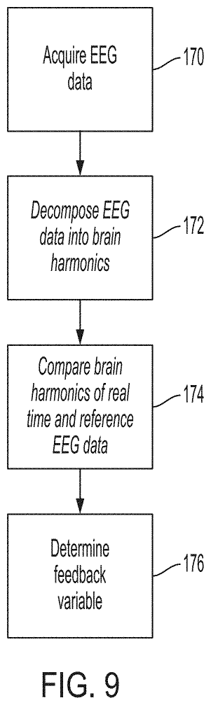

According to the second approach, referred to as “Harmonic Decomposition Comparison,” both the reference EEG data and the real-time EEG data 16 are decomposed into brain harmonics generated by the brain harmonic estimation unit 120 using a harmonic brain state decomposition technique. As shown for example in FIGS. 3, 9 and 10, the brain data, such as real-time EEG data 16, and the reference EEG data can be conveyed to the feedback variable generation unit 20, step 170. The real-time EEG data 16 is decomposed in real-time using a harmonic brain state decomposition method or technique to estimate the relative contributions of each harmonic brain state, steps 172 and 202. For the real-time EEG data 16, the decomposition is performed in real-time thus leading to the contribution of each of the brain's harmonics needed to reconstruct the current brain activity measured in real-time, step 204. The harmonic contributions estimated in real-time from the real time EEG data are then compared to the contributions of the brain's harmonics in the reference EEG data acquired in a desired mental state, step 174. A distance between the brain harmonics is then computed by applying a distance measuring technique to the two sets of harmonic contributions to yield a distance computation, step 206. The resulting distance, called a harmonic contribution distance, is then used to determine the value of the feedback variable 22, steps 176 and 208.

The illustrated neurofeedback system 70 can include an optional mapping unit, such as the mapping unit 40 of FIG. 3, to map the feedback variable 22 to one or more selected sensory feedback devices forming part of the sensory feedback unit 50. Specifically, the neurofeedback can be estimated based on the feedback variable 22, step 198. The selected feedback device forming part of the sensory feedback unit 50 can then be selected, and the neurofeedback can be applied to the subject 12, step 200.

Further, the feedback variable 22 or the output of the mapping unit 40 of the neurofeedback system 70 can be processed by an optional classification unit 44 that can estimate or determine the subject's specific mental state based on the feedback signal or the mapping unit signal. Further, the classification unit 44 can optionally estimate or determine a desired mental state that the subject needs to attain in order to best address the actual mental state of the subject. The desired mental state can be calculated or determined by the classification unit 44 from the information associated with the EEG signals and the feedback variable, or the desired mental state can be preselected by a medical professional based on a subject's assessment.

In this approach, the harmonic brain state decomposition is performed in real-time. The previously computed brain harmonics 132A can be chosen to be estimated in various different ways, such as dense structural harmonics, parcellated structural harmonics, dense functional harmonics, spherical harmonics mapped to the shape of the cortex or the scalp, as well as EEG harmonics introduced for the first time in this intervention.

Both approaches or techniques, the Direct Harmonic Comparison approach and the Harmonic Decomposition Comparison approach, incorporate harmonic brain states to represent EEG data in a neurofeedback system, and only differ in that the feedback is estimated from harmonic brain states. In the former approach, the feedback is estimated by comparing the EEG brain harmonics directly (e.g. their shapes, any topological measure applied to their shapes or their corresponding eigenvalues), whereas in the latter approach the feedback is computed using the comparison of the harmonic decompositions of the EEG reference and real-time EEG signals (e.g. using their contribution, the power of their contribution or the energy of their contribution or any measure applied to their relative contributions). Thus, by incorporating the use of harmonic brain states the neurofeedback system of the present invention offers various advantage over existing system.

The harmonic brain state estimation and decomposition of the EEG data into the harmonic brain states also allows the neurofeedback system 70 to represent brain activity measured by EEG as brain states defined on the whole brain, or cortex, or scalp or across the whole set of EEG electrodes, thus incorporating a more complete picture of the nature of brain activity into the neurofeedback systems. A subject's difference from the neurotypical population can be easily calculated, and significant harmonic brain state differences can be identified either by comparing the shape of the estimated harmonic brain states directly or by comparing derived measures applied on their relative contributions that are estimated by decomposing the EEG signal into the harmonic brain states.

Thus, it is a primary objective of this disclosure to provide a neurofeedback system, process, and method of use for administering superior neurofeedback by capturing EEG brain activity, using various electrodes at optimal locations, analyzing data, and estimating the harmonic brain states (brain's harmonics) using the current pattern of brain activity or by decomposing the current pattern of brain activity into harmonic brain states (brain harmonics) that have been previously estimated using a suitable connectivity matrix representing the brain's connectivity, in order to aid the subject by guiding them to regulate their brain activity through the provided feedback, which aims at closing the gap between the measured brain activity and desired brain activity. The present invention allows for the integration of optional classification methods including but not limited to machine learning or artificial intelligence to either harmonic shape distance or harmonic contribution distance to classify the mental state of the subject (e.g., anxious, depressed, neurotypical, etc.)

The neurofeedback system of the present invention 70 may employ artificial intelligence or machine learning techniques, such as neural networks, to classify the mental states based on the harmonic brain states. The neurofeedback system of the present invention may include machine-readable media for electronic management, analysis, recreation, publication, and distribution of the data gathered by the neurofeedback system. The neurofeedback system thus presents an example of a system suitable for the gathering, viewing, editing, and analyzing EEG data and prescribing actions based on the analysis of the data.

In one example, the system disclosed herein is a neurofeedback system, process, and method of use for a 19-channel (or more) EEG device for the multi-channel EEG measurement and analog to digital conversion of the measured data. Additionally, the system can be implemented in other EEG devices with a fewer or more channels depending on the desired accuracy.

Additionally, the system can have various digital implementations, including implementation within firmware of an EEG device or in a computing device having suitable software and hardware during or after the EEG signal digitization. Any digital implementation of the electronic system may include storing, processing, or replacing data or using databases or datasets as a source for creating information. In other words, the process and method disclosed herein capture EEG data using a plurality of electrodes distributed over the scalp.

The data gathered from the electrodes is then represented using the harmonic brain states by implementing one of the two alternative methodologies for estimating the feedback variable. Thus, the harmonic signatures or brain states found in the real-time EEG data of the subject receiving the neurofeedback can be compared to the subject's harmonic signatures or brain states previously estimated in various mental states, such as anxious, depressed, relaxed, neutral, focused, distracted, asleep, and the like. The differences between the current real-time brain state of the subject and the subject's choice of the desired mental state (e.g., neurotypical relaxed) can be used to trigger sensory feedback action, which guides the user. The system of the present invention can also provide a neurofeedback system, process, and method of use that captures and processes brain data and can learn the unique brain waves or brain activity patterns of a user (harmonic brain states) and then determine when that particular user has a specific mental state or has entered an altered mental state which is undesired.

Other objectives of the disclosure are to provide a neurofeedback system, process, and method of use that provides a sensory cue that can train the brain to reach a desired mental state, regulate the brain activity, deliver EEG neurofeedback, store EEG brainwave data, track improvements over time, determine how different variables affect a user's mental state, track the average mental state of a user, enable a user to monitor brain activity in real-time, generate a flow state for a user, provide operant conditioning, provide a user interface, improve upon EEG sensing.

The neurofeedback cycle of FIGS. 3 and 8-10 can be continuously repeated during a session. The length of the session is determined by the professional applying the feedback protocol, the subject's availability, and other relevant health and practical factors. In another embodiment, the session can also be constructed to provide feedback on event-related potentials (ERPs). In such a setting, the difference between the desired ERPs (correspondingly the associated harmonic brain state activations) and the subject's measured ERPs can be measured and utilized in the feedback variable calculation.

The present invention is thus directed to a neurofeedback system and associated method that determines the harmonic brain states of a subject from input brain data, which can include EEG data, reference EEG data (Laplace eigenvectors and eigenvalues), and MRI type data (e.g., MRI data and fMRI data). Regarding estimating or determining the harmonic brain states, the neurofeedback system 70 can form a data pipeline that represents brain activity as measured by sensed EEG data of a subject acquired in various mental states. For example, the mental states of the subject can include resting state or during task-performance or event-related potentials (EPRs). Using a Laplace eigenfunction technique, these harmonic brain states can be estimated from the input EEG data as harmonic modes. This technique can process brain connectivity data (e.g., structural and functional brain connectivity data) generated from input EEG data and/or Mill type data (e.g., fMRI data), with or without using any parcellation method. Furthermore, the present invention is also directed to determining or estimating the harmonic brain states directly from the EEG data (e.g., Direct Harmonic Comparison technique).

Further, the harmonic brain states can be determined by decomposing the input brain data (e.g., Harmonic Decomposition technique). The neurofeedback system thus either uses the estimated EEG brain harmonics directly to compare the signatures of the real-time EEG data to the reference EEG data or decomposes the EEG data in real-time into harmonic brain states by estimating each of the harmonic brain states' contribution for reconstructing the pattern of brain activity estimated from the EEG data.

The neurofeedback system of the present invention can also be employed to classify the mental state of the subject. The system can perform this classification via a classification unit by using the activation profile (contributions) of harmonic brain states or by analyzing the topological properties of the real-time EEG harmonics.

The system can also determine or estimate the appropriate feedback to the subject. The neurofeedback pipeline can include the administration of neurofeedback based on the difference between the activation profile (contributions) of harmonic brain states of the current pattern of brain activity estimated from the current EEG measurements in real-time and that of the desired mental state (e.g., harmonic brain state activation profile of EEG measurements in a relaxed state).

The neurofeedback system of the present invention forms a neurofeedback paradigm that incorporates brain harmonics into the feedback pipeline, and therefore inherently respects the intrinsic relations in brain function. Furthermore, by introducing the concept of harmonic brain states into the systems, the present invention considers the brain activity pattern across the whole cortex or scalp and not only that of a region of interest (ROI).

The mental state classification can be performed by first finding the inverse solution of the measured EEG signals, which can be used to calculate the EEG harmonic decomposition. A machine learning classifier unit can process the contribution/weight/activation of the harmonic brain states estimated by the EEG harmonic decomposition technique to determine the mental state classification.

It will be appreciated by those skilled in the art that other various modifications could be made to the process and method of use without parting from the spirit and scope of this disclosure. All such modifications and changes fall within the scope of the claims and are intended to be covered thereby. The neurofeedback system, process, and method of use, disclosed herein, may be formed of any suitable size, shape, and design and is configured to help a user in improving his/her mental state. The disclosure herein is configured to integrate with various platforms and/or accept various input from a plurality of sensors. By taking data from multiple inputs and examining that data over time, accuracy is increased, and individual and varying behaviors can be accommodated. In this way, the disclosure herein can aid a user in improving their mental health. Additionally, the disclosure herein can train a user over the long-term and provide various cues and training methodologies.

Embodiments of the present disclosure may include systems and methods that automatically pull all EEG and other information, such as pulse, body temperature, and other sensed data, into a comprehensive database containing a myriad of information about the individual. This data, combined with age, sex, and additional information, can allow a better neurofeedback protocol selection for the specific user.

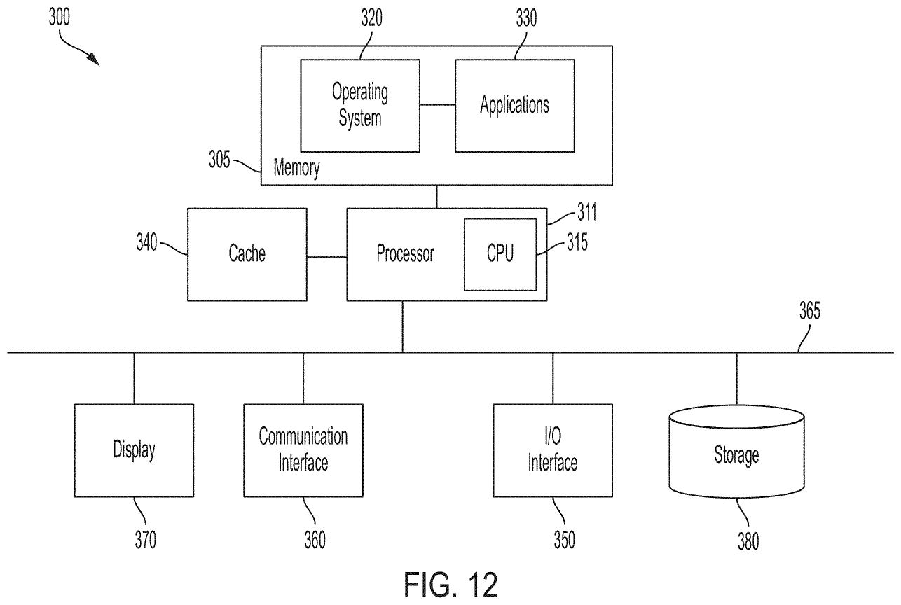

It should be appreciated that various concepts, systems and methods described above can be implemented in any number of ways, as the disclosed concepts are not limited to any particular manner of implementation or system configuration. Examples of specific implementations and applications are discussed below and shown in FIG. 12 primarily for illustrative purposes and for providing or describing the operating environment of the system of the present invention. The neurofeedback systems 10 and 70 and elements, modules and units thereof can employ one or more electronic or computing devices, such as one or more servers, clients, computers, laptops, smartphones and the like, that are networked together or which are arranged so as to effectively communicate with each other. The network can be any type or form of network. The devices can be on the same network or on different networks. In some embodiments, the network system may include multiple, logically-grouped servers. In one of these embodiments, the logical group of servers may be referred to as a server farm or a machine farm. In another of these embodiments, the servers may be geographically dispersed. The electronic devices can communicate through wired connections or through wireless connections. The clients can also be generally referred to as local machines, clients, client nodes, client machines, client computers, client devices, endpoints, or endpoint nodes. The servers can also be referred to herein as servers, server nodes, or remote machines. In some embodiments, a client has the capacity to function as both a client or client node seeking access to resources provided by a server or server node and as a server providing access to hosted resources for other clients. The clients can be any suitable electronic or computing device, including for example, a computer, a server, a smartphone, a smart electronic pad, a portable computer, and the like, such as the electronic or computing device 300. The present invention can employ one or more of the illustrated computing devices and can form a computing system. Further, the server may be a file server, application server, web server, proxy server, appliance, network appliance, gateway, gateway server, virtualization server, deployment server, SSL VPN server, or firewall, or any other suitable electronic or computing device, such as the electronic device 300. In one embodiment, the server may be referred to as a remote machine or a node. In another embodiment, a plurality of nodes may be in the path between any two communicating servers or clients. The neurofeedback system which includes the spatial distribution determination unit, the feedback variable generation unit, the mapping unit, brain connectivity estimation unit, brain harmonic estimation unit and the like (“elements of the system”) can be stored on one or more of the clients or servers, and the hardware associated with the client or server, such as the processor or CPU and memory described below.

FIG. 12 is a high-level block diagram of an electronic or computing device 300 that can be used with the embodiments disclosed herein. Without limitation, the hardware, software, and techniques described herein can be implemented in digital electronic circuitry or in computer hardware that executes firmware, software, or combinations thereof. The implementation can include a computer program product (e.g., a non-transitory computer program tangibly embodied in a machine-readable storage device, for execution by, or to control the operation of, one or more data processing apparatuses, such as a programmable processor, one or more computers, one or more servers and the like).

The illustrated electronic device 300 can be any suitable electronic circuitry that includes a main or central memory unit 305 that is connected to a processor 311 having a CPU 315 and a cache unit 340 configured to store copies of the data from the most frequently used main memory 305. The electronic device can implement the process flow identification system 10 or one or more elements of the process flow identification system.

Further, the methods and procedures for carrying out the methods disclosed herein can be performed by one or more programmable processors executing a computer program to perform functions of the invention by operating on input data and generating output. Further, the methods and procedures disclosed herein can also be performed by, and the apparatus disclosed herein can be implemented as, special purpose logic circuitry, such as a FPGA (field-programmable gate array) or an ASIC (application-specific integrated circuit). Modules and units disclosed herein can also refer to portions of the computer program and/or the processor/special circuitry that implements that functionality.

The processor 311 is any logic circuitry that responds to, processes or manipulates instructions received from the main memory unit, and can be any suitable processor for execution of a computer program. For example, the processor 311 can be a general or special purpose microprocessor or a processor of a digital computer. The CPU 315 can be any suitable processing unit known in the art. For example, the CPU 315 can be a general or special purpose microprocessor, such as an application-specific instruction set processor, graphics processing unit, physics processing unit, digital signal processor, image processor, coprocessor, floating-point processor, network processor, and/or any other suitable processor that can be used in a digital computing circuitry. Alternatively or additionally, the processor can comprise at least one of a multi-core processor and a front-end processor. Generally, the processor 311 can be embodied in any suitable manner. For example, the processor 311 can be embodied as various processing means such as a microprocessor or other processing element, a coprocessor, a controller or various other computing or processing devices including integrated circuits such as, for example, an ASIC (application-specific integrated circuit), an FPGA (field-programmable gate array), a hardware accelerator, or the like.

Additionally or alternatively, the processor 311 can be configured to execute instructions stored in the memory 305 or otherwise accessible to the processor 311. As such, whether configured by hardware or software methods, or by a combination thereof, the processor 311 can represent an entity (e.g., physically embodied in circuitry) capable of performing operations according to embodiments disclosed herein while configured accordingly. Thus, for example, when the processor 311 is embodied as an ASIC, FPGA or the like, the processor 311 can be specifically configured hardware for conducting the operations described herein. Alternatively, as another example, when the processor 311 is embodied as an executor of software instructions, the instructions can specifically configure the processor 311 to perform the operations described herein. In many embodiments, the central processing unit 315 is provided by a microprocessor unit, e.g.: those manufactured by Intel Corporation of Mountain View, Calif.; those manufactured by Motorola Corporation of Schaumburg, Ill.; the ARM processor and TEGRA system on a chip (SoC) manufactured by Nvidia of Santa Clara, Calif.; the POWER7 processor, those manufactured by International Business Machines of White Plains, N.Y.; or those manufactured by Advanced Micro Devices of Sunnyvale, Calif. The processor can be configured to receive and execute instructions received from the main memory 305.

The electronic device 300 applicable to the hardware of the present invention can be based on any of these processors, or any other processor capable of operating as described herein. The central processing unit 315 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

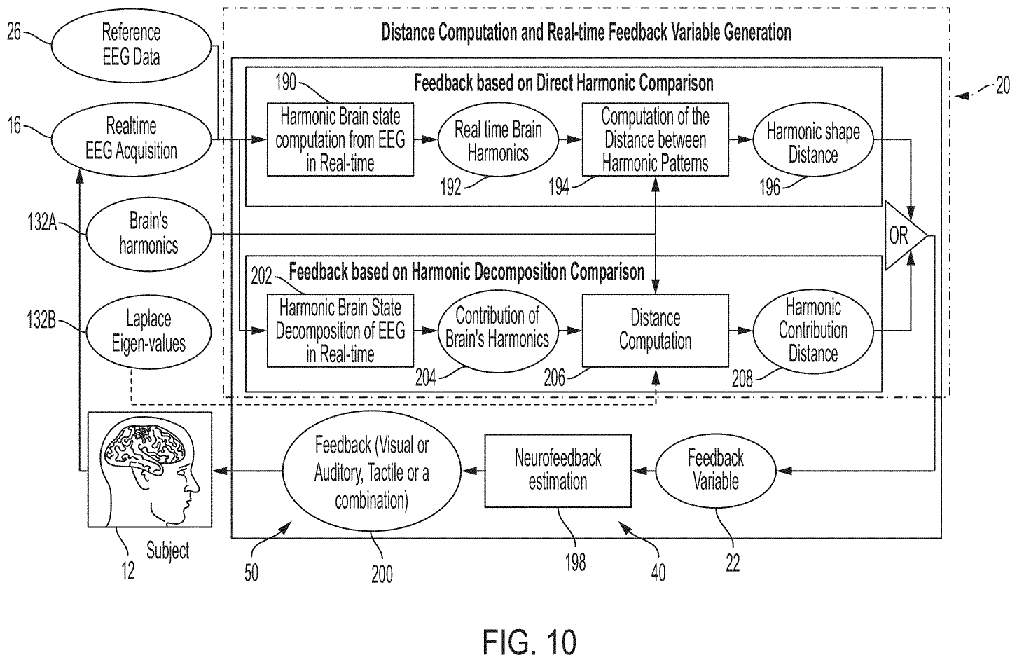

The processor 311 and the CPU 315 can be configured to receive instructions and data from the main memory 305 (e.g., a read-only memory or a random access memory or both) and execute the instructions. The instructions and other data can be stored in the main memory 305. The processor 311 and the main memory 305 can be included in or supplemented by special purpose logic circuitry. The main memory unit 305 can include one or more memory chips capable of storing data and allowing any storage location to be directly accessed by the processor 311. The main memory unit 305 may be volatile and faster than other memory in the electronic device, or can dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 305 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 305 can be based on any of the above described memory chips, or any other available memory chips capable of operating as described herein. In the embodiment shown in FIG. 10, the processor 311 communicates with main memory 305 via a system bus 365. The computer executable instructions of the present invention may be provided using any computer-readable media that is accessible by the computing or electronic device 300. Computer-readable media may include, for example, the computer memory or storage unit 305. The computer storage media may also include, but is not limited to, RAM, ROM, EPROM, EEPROM, flash memory or other memory technology, CD-ROM, digital versatile disks (DVD) or other optical storage, magnetic cassettes, magnetic tape, magnetic disk storage or other magnetic storage devices, or any other non-transmission medium that can be used to store information for access by a computing device.

In contrast, communication media may embody computer readable instructions, data structures, program modules, or other data in a modulated data signal, such as a carrier wave, or other transport mechanism. As defined herein, computer readable storage media does not include communication media. Therefore, a computer storage or memory medium should not be interpreted to be a propagating signal per se or stated another transitory in nature. The propagated signals may be present in a computer storage media, but propagated signals per se are not examples of computer storage media, which is intended to be non-transitory. Although the computer memory or storage unit 305 is shown within the computing device 300 it will be appreciated that the storage may be distributed or located remotely and accessed via a network or other communication link.

The main memory 305 can comprise an operating system 320 that is configured to implement various operating system functions. For example, the operating system 320 can be responsible for controlling access to various devices, memory management, and/or implementing various functions of the asset management system disclosed herein. Generally, the operating system 320 can be any suitable system software that can manage computer hardware and software resources and provide common services for computer programs.

The main memory 305 can also hold application software 330. For example, the main memory 305 and application software 330 can include various computer executable instructions, application software, and data structures, such as computer executable instructions and data structures that implement various aspects of the embodiments described herein. For example, the main memory 305 and application software 330 can include computer executable instructions, application software, and data structures, such as computer executable instructions and data structures that implement various aspects of the content characterization systems disclosed herein, such as processing and capture of information. Generally, the functions performed by the systems disclosed herein can be implemented in digital electronic circuitry or in computer hardware that executes software, firmware, or combinations thereof. The implementation can be as a computer program product (e.g., a computer program tangibly embodied in a non-transitory machine-readable storage device) for execution by or to control the operation of a data processing apparatus (e.g., a computer, a programmable processor, or multiple computers). Generally, the program codes that can be used with the embodiments disclosed herein can be implemented and written in any form of programming language, including compiled or interpreted languages, and can be deployed in any form, including as a stand-alone program or as a component, module, subroutine, or other unit suitable for use in a computing environment. A computer program can be configured to be executed on a computer, or on multiple computers, at one site or distributed across multiple sites and interconnected by a communications network, such as the Internet.

The processor 311 can further be coupled to a database or data storage 380. The data storage 380 can be configured to store information and data relating to various functions and operations of the content characterization systems disclosed herein. For example, as detailed above, the data storage 380 can store information including but not limited to captured information, multimedia, processed information, and characterized content.

Many I/O devices may be present in or connected to the electronic device 300. For example, the electronic device can include a display 370, and as previously described, the visual application unit 28 or one or more other elements of the system 10 can include the display. The display 370 can be configured to display information and instructions received from the processor 311. Further, the display 370 can generally be any suitable display available in the art, for example a Liquid Crystal Display (LCD), a light emitting diode (LED) display, digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays, or electronic papers (e-ink) displays. Furthermore, the display 370 can be a smart and/or touch sensitive display that can receive instructions from a user and forwarded the received information to the processor 311. The input devices can also include user selection devices, such as keyboards, mice, trackpads, trackballs, touchpads, touch mice, multi-touch touchpads, touch mice and the like, as well as microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. The output devices can also include video displays, graphical displays, speakers, headphones, inkjet printers, laser printers, and 3D printers.

The electronic device 300 can also include an Input/Output (I/O) interface 350 that is configured to connect the processor 311 to various interfaces via an input/output (I/O) device interface 380. The device 300 can also include a communications interface 360 that is responsible for providing the circuitry 300 with a connection to a communications network (e.g., communications network 120). Transmission and reception of data and instructions can occur over the communications network.

It is also to be understood that although the invention has been described above in terms of particular embodiments, the foregoing embodiments are provided as illustrative only, and do not limit or define the scope of the invention. Various other embodiments, including but not limited those described herein are also within the scope of the claims. For example, elements, units, tools and components described herein may be further divided into additional components or joined together to form fewer components for performing the same functions.

Any of the functions disclosed herein may be implemented using means for performing those functions. Such means include, but are not limited to, any of the components disclosed herein, such as the electronic or computing device components described herein.

The techniques described above may be implemented, for example, in hardware, one or more computer programs tangibly stored on one or more computer-readable media, firmware, or any combination thereof. The techniques described above may be implemented in one or more computer programs executing on (or executable by) a programmable computer including any combination of any number of the following: a processor, a storage medium readable and/or writable by the processor (including, for example, volatile and non-volatile memory and/or storage elements), an input device, and an output device. Program code may be applied to input entered using the input device to perform the functions described and to generate output using the output device.

The term electronic device or computing device can refer to any device that includes a processor and a computer-readable memory capable of storing computer-readable instructions, and in which the processor is capable of executing the computer-readable instructions in the memory. The terms computer system and computing system refer herein to a system containing one or more computing devices.

Embodiments of the present invention include features which are only possible and/or feasible to implement with the use of one or more computers, computer processors, and/or other elements of a computer system. Such features are either impossible or impractical to implement mentally and/or manually. For example, embodiments of the present invention may operate on digital electronic processes which can only be created, stored, modified, processed, and transmitted by computing devices and other electronic devices. Such embodiments, therefore, address problems which are inherently computer-related and solve such problems using computer technology in ways which could not be solved manually or mentally by humans.

Any claims herein which affirmatively require a computer, a processor, a memory, or similar computer-related elements, are intended to require such elements, and should not be interpreted as if such elements are not present in or required by such claims. Such claims are not intended, and should not be interpreted, to cover methods and/or systems which lack the recited computer-related elements. For example, any method claim herein which recites that the claimed method is performed by a computer, a processor, a memory, and/or similar computer-related element, is intended to, and should only be interpreted to, encompass methods which are performed by the recited computer-related element(s). Such a method claim should not be interpreted, for example, to encompass a method that is performed mentally or by hand (e.g., using pencil and paper). Similarly, any product claim herein which recites that the claimed product includes a computer, a processor, a memory, and/or similar computer-related element, is intended to, and should only be interpreted to, encompass products which include the recited computer-related element(s). Such a product claim should not be interpreted, for example, to encompass a product that does not include the recited computer-related element(s).

Embodiments of the present invention solve one or more problems that are inherently rooted in computer technology. For example, embodiments of the present invention solve the problem of how to associate a technical artifact with a node of a process flow diagram. There is no analog to this problem in the non-computer environment, nor is there an analog to the solutions disclosed herein in the non-computer environment.

Furthermore, embodiments of the present invention represent improvements to computer and communication technology itself. For example, the system 10 of the present can optionally employ a specially programmed or special purpose computer in an improved computer system, which may, for example, be implemented within a single computing device.

Each computer program within the scope of the claims below may be implemented in any programming language, such as assembly language, machine language, a high-level procedural programming language, or an object-oriented programming language. The programming language may, for example, be a compiled or interpreted programming language.

Each such computer program may be implemented in a computer program product tangibly embodied in a machine-readable storage device for execution by a computer processor. Method steps of the invention may be performed by one or more computer processors executing a program tangibly embodied on a computer-readable medium to perform functions of the invention by operating on input and generating output. Suitable processors include, by way of example, both general and special purpose microprocessors. Generally, the processor receives (reads) instructions and data from a memory (such as a read-only memory and/or a random access memory) and writes (stores) instructions and data to the memory. Storage devices suitable for tangibly embodying computer program instructions and data include, for example, all forms of non-volatile memory, such as semiconductor memory devices, including EPROM, EEPROM, and flash memory devices; magnetic disks such as internal hard disks and removable disks; magneto-optical disks; and CD-ROMs. Any of the foregoing may be supplemented by, or incorporated in, specially-designed ASICs (application-specific integrated circuits) or FPGAs (Field-Programmable Gate Arrays). A computer can generally also receive (read) programs and data from, and write (store) programs and data to, a non-transitory computer-readable storage medium such as an internal disk (not shown) or a removable disk. These elements can also be found in a conventional desktop or workstation computer as well as other computers suitable for executing computer programs implementing the methods described herein, which may be used in conjunction with any digital print engine or marking engine, display monitor, or other raster output device capable of producing color or gray scale pixels on paper, film, display screen, or other output medium.

Any data disclosed herein may be implemented, for example, in one or more data structures tangibly stored on a non-transitory computer-readable medium. Embodiments of the invention may store such data in such data structure(s) and read such data from such data structure(s).

We claim:

1. A method for determining a type of sensory feedback to be applied to a subject, comprising
acquiring brain data,
estimating from the brain data a plurality of harmonic brain states,
determining a feedback variable based on the plurality of harmonic brain states,
mapping the feedback variable to one or more types of sensory feedback, and
applying the sensory feedback to the subject with one or more types of sensory feedback devices.

2. The method of claim 1, further comprising estimating a mental state of the subject based on the plurality of brain states and the feedback variable.

3. The method of claim 1, further comprising determining a brain connectivity matrix data indicative of a connectivity of the brain based on the brain data.

4. The method of claim 3, wherein the brain data includes MRI and DTI type data, and wherein the step of determining the brain connectivity matrix data comprises
applying one or more structural connectivity techniques to the MRI data to estimate physical gray matter connections or white-matter inter-regional pathways in the brain based on the MRI and DTI type data, respectively, and
generating output structural brain connectivity data.

5. The method of claim 5, further comprising applying a parcellation technique to the structural brain connectivity data to generate output parcellated brain connectivity data.

6. The method of claim 3, wherein the brain data includes functional MM type data, and wherein the step of determining the brain connectivity matrix data comprises
applying one or more functional connectivity techniques to the functional MRI type data to estimate functional interrelationships between regions of the brain, and
generating output functional brain connectivity data.

7. The method of claim 6, further comprising applying a parcellation technique to the output functional brain connectivity data to generate output parcellated brain connectivity data.

8. The method of claim 3, wherein the brain data includes EEG data, and wherein the step of determining the brain connectivity matrix data comprises
applying one or more functional connectivity techniques to the EEG data,
mapping the EEG data onto a cortical surface of the subject using a source location technique,
estimating a functional connectivity of the brain by performing temporal correlations of the brain data over time, and
generating output functional brain connectivity data.

9. The method of claim 8, further comprising applying a parcellation technique to the functional brain connectivity data to generate output parcellated brain connectivity data.

10. The method of claim 3, wherein the brain data includes EEG data, and wherein the step of determining the brain connectivity matrix data comprises
applying one or more functional connectivity techniques to the EEG data,
estimating a functional connectivity of the brain based on the EEG data free of source localization, and
generating output signal connectivity data.

11. The method of claim 3, wherein the step of estimating the plurality of harmonic brain states comprises determining, using a Laplace eigendecomposition technique, the plurality of harmonic brain states based on the brain connectivity matrix data.

12. The method of claim 11, wherein determining the plurality of harmonic brain states comprises
determining an adjacency matrix data based on the brain connectivity matrix data,
determining a graph Laplacian matrix data based on the adjacent matrix data, and
decomposing the graph Laplacian matrix data to estimate the plurality of harmonic brain states.

13. The method of claim 11, wherein determining an adjacency matrix based on the brain connectivity matrix data comprises determining the adjacency matrix using a K-nearest neighbor technique or an epsilon-balls technique.

14. The method of claim 12, wherein decomposing the graph Laplacian matrix data to estimate the plurality of harmonic brain states comprises
generating a plurality of eigenvectors indicative of the plurality of harmonic brain states, and
generating a plurality of eigenvalues associated with the plurality of eigenvectors.

15. The method of claim 14, wherein the plurality of harmonic brain states forms a plurality of reference harmonic brain states, wherein determining a feedback variable based on the plurality of harmonic brain states comprises
acquiring real-time EEG data,
determining directly a plurality of EEG harmonic brain states associated with the EEG data,
determining a distance between the plurality of EEG harmonic brain states and the plurality of reference harmonic brain states using a distance measuring technique to form a harmonic distance, and
determining the feedback variable based on the harmonic distance.

16. The method of claim 14, wherein the plurality of harmonic brain states forms a plurality of reference harmonic brain states, wherein determining a feedback variable based on the plurality of harmonic brain states comprises
acquiring real-time EEG data,
determining a plurality of harmonic brain states based on MRI, DTI, fMRI or EEG data prior to real-time EEG acquisition,
decomposing the EEG data using a harmonic brain state decomposition technique to estimate a relative contribution of each of the plurality of harmonic brain states to the EEG data,
determining a distance between the plurality of harmonic brain states' contributions to the real-time and reference EEG using a distance measuring technique to form a harmonic contribution distance, and
determining the feedback variable based on the harmonic contribution distance.

17. The method of claim 1, further comprising reconstructing brain activity of the subject from the plurality of harmonic brain states.

18. A system for determining a type of sensory feedback to be applied to a subject, comprising
a brain connectivity estimation unit for estimating a brain connectivity matrix data indicative of a connectivity of the brain based on input brain data,
a brain harmonic estimation unit for determining from the brain connectivity matrix data a plurality of harmonic brain states, and
a feedback variable generation unit for determining a feedback variable based on the plurality of harmonic brain states.

19. The system of claim 18, further comprising a mapping unit for mapping the feedback variable to one or more types of sensory feedback devices of a sensory feedback unit, wherein the sensory feedback device is configured to apply a sensory feedback to the subject

20. The system of claim 19, further comprising a classification unit for estimating a mental state of the subject based on the plurality of brain states and the feedback variable.

21. The system of claim 18, wherein the brain data includes MRI and DTI type data, and wherein the brain connectivity estimation unit comprises a structural connectivity unit for applying one or more structural connectivity techniques to the MRI and DTI type data to estimate physical gray-matter connections and physical white-matter inter-regional pathways in the brain based on the MM and DTI type data, respectively, and for generating output structural brain connectivity data.

22. The system of 21, wherein the brain connectivity estimation unit further comprises a parcellation unit for applying a parcellation technique to the structural brain connectivity data to generate output parcellated brain connectivity data.

23. The system of claim 18, wherein the brain data includes functional Mill type data, and wherein the brain connectivity estimation unit comprises a functional connectivity unit for applying one or more functional connectivity techniques to the functional Mill type data to estimate functional interrelationships between regions of the brain and for generating output functional brain connectivity data.

24. The system of claim 23, wherein the brain connectivity estimation unit further comprises a parcellation unit for applying a parcellation technique to the output functional brain connectivity data to generate output parcellated brain connectivity data.

25. The system of claim 18, wherein the brain data includes EEG data, and wherein the brain connectivity estimation unit comprises a functional connectivity unit configured for:
applying one or more functional connectivity techniques to the EEG data,
mapping the EEG data onto a cortical surface of the subject using a source localization technique,
estimating a functional connectivity of the brain by performing temporal correlations of the brain data over time, and
generating output functional brain connectivity data.

26. The system of claim 25, wherein the brain connectivity estimation unit further comprises a parcellation unit for applying a parcellation technique to the functional brain connectivity data to generate output parcellated brain connectivity data.

27. The system of claim 18, wherein the brain data includes EEG data, and wherein the brain connectivity estimation unit comprises a functional connectivity unit configured for:
applying one or more functional connectivity techniques to the EEG data,
estimating a functional connectivity of the brain based on the EEG data free of source localization, and
generating output signal connectivity data.

28. The system of claim 18, wherein the brain harmonic estimation unit comprises an adjacency matrix unit for generating adjacency matrix data based on the brain connectivity matrix data.

29. The system of claim 28, wherein the brain harmonic estimation unit further comprises a graph Laplacian determination unit for generating, based on the adjacency matrix data, graph Laplacian matrix data.

30. The system of claim 29, wherein the brain harmonic estimation unit further comprises a decomposition unit for decomposing the graph Laplacian matrix data and for generating Laplace eigen vectors representative of the plurality of harmonic brain states and corresponding eigenvalues.

31. The system of claim 18, wherein the plurality of harmonic brain states forms a plurality of reference harmonic brain states, wherein the feedback variable generation unit is configured to:
directly determine a plurality of EEG harmonic brain states from real-time EEG data,
determine a distance between the plurality of EEG harmonic brain states and the plurality of reference harmonic brain states using a distance measuring technique to form a harmonic distance, and
determine the feedback variable based on the harmonic distance.

32. The system of claim 18, wherein the plurality of harmonic brain states forms a plurality of reference harmonic brain states, and wherein the feedback variable generation unit is configured to:
decompose real-time EEG data using a harmonic brain state decomposition technique to determine a plurality of harmonic brain states previously estimated from MRI, DTI, fMRI or EEG data in order to estimate a relative contribution of each of the plurality of harmonic brain states to the measured EEG data,
determine a distance between the plurality of harmonic brain states' contributions to the real-time EEG data and the plurality of harmonic brain states' contributions to reference EEG data using a distance measuring technique to form a harmonic contribution distance, and
determine the feedback variable based on the harmonic contribution distance.
